(19) United States
(12) Patent Application Publication
(10) Pub. No.: US 2019/0143118 A1
(43) Pub. Date: May 16, 2019

(54) LEADS AND METHODS FOR CARDIAC RESYNCHRONIZATION THERAPY

(71) Applicant: John R. BULLINGA, Haverford, PA (US)

(72) Inventor: John R. BULLINGA, Haverford, PA (US)

(21) Appl. No.: 16/099,137

(22) PCT Filed: May 4, 2017
(86) PCT No.: PCT/US17/31123

§ 371 Date: Nov. 5, 2018

Related U.S. Application Data
(60) Provisional application No. 62/331,885, filed on May 4, 2016.

(51) Int. Cl. A61N 1/368; A61N 1/08; A61N 1/05

(52) U.S. Cl. CPC A61N 1/3686; A61N 1/3627; A61N 1/056; A61N 1/08

(57) ABSTRACT

The present invention relates to devices and methods used in cardiac resynchronization therapy. Novel cardiac leads for the right and left ventricles are disclosed. Also disclosed is a method of stimulating the heart using pulse sequences that excite the heart using a plurality of ventricular leads while reducing energy consumption by delivering pulses to the electrodes in an overlapping multiphasic manner.

### RELATED APPLICATION

The present application claims priority to and the benefit of U.S. patent application 62/331,885, “Leads and Methods for Cardiac Resynchronization Therapy” (filed on May 4, 2016), the entirety of which application is incorporated herein for any and all purposes.

### FIELD OF THE INVENTION

Disclosed herein are methods and devices relating to cardiac resynchronization therapy.

### BACKGROUND OF THE INVENTION

Cardiac resynchronization therapy (CRT) with multisite pacing is an effective treatment for heart failure in patients with left ventricular dyssynchrony. A CRT system may comprise a pulse generator that includes electronics and a battery and leads connected proximally to the pulse generator and distally attached to the heart. Although biventricular pacing has become a standard approach for CRT, its efficacy has substantial variability, and 30-50% of patients have no significant improvement in ventricular function in response to currently available CRT approaches. Patients may be nonresponsive to these CRT approaches because of inadequate resynchronization due to the relative position of electrodes, the severity of cardiomyopathy, ventricular dilatation, or a combination thereof. In some patients with severe cardiomyopathy, even when leads are in ideal positions, dual site pacing is inadequate to produce a significant improvement in ventricular function. When leads are in sub-ideal positions for dual site pacing, ventricular function may remain at undesirable levels because of incomplete resynchronization. Important goals for CRT methods are to improve patients' response rates and ventricular function.

A major problem with pacing from multiple electrodes is that there is an incremental current drain with each electrode being paced. Incremental current drain refers to the continuous discharge from a battery to each electrode in a pacing system. This increased current drain limits the pulse generator battery longevity. Each time a pulse generator, or pulse generator battery, change is necessary, invasive techniques are employed for which there are associated financial costs and risks of infection at the pulse generator implantation site. It is highly desirable to pace from multiple electrodes while reducing (or even minimizing) the current drain on the pulse generator.

Thus, there is a long-felt need for cardiac leads that may be deployed in the heart with an improved electrode distribution that allows for improved cardiac resynchronization. There is also a long-felt need exists for devices and methods that allow for effective pacing of multiple sites in the heart while also reducing (or even minimizing) battery drain on the pulse generator.

### SUMMARY OF THE INVENTION

Disclosed herein are devices for a multi-site right ventricular lead, comprising: a distal section that is elongate along a central axis, the distal section having a terminus and having a distal fixation mechanism that extends away from the distal terminus; and a proximal section that is elongate along the central axis and in electrical communication with the distal section, the proximal section having at least one electrode and an end adapted to be connected to a pulse generator.

Also disclosed herein are devices for a left ventricular lead system, comprising an outer lead that is elongate along a central axis, the outer lead having a first end adapted to be connected to a pulse generator, a second end having at least one electrode; a first lumen that is disposed along or parallel to the central axis and between the first end and the second end of the outer lead and configured to receive a connector, the first lumen having a receiving port; and a second lumen configured to house a portion of the inner lead, the second lumen being disposed along or parallel to the central axis. This system also comprises an inner lead having a first end adapted to be connected to the receiving port, a second end having at least one electrode; and a securing mechanism disposed at or near the second lumen, the securing mechanism being configured to prevent the inner lead from moving in the second lumen once activated. Preventing the inner lead from moving through the second lumen reduces the likelihood of the lead becoming displaced from the cardiac tissue

Also provided are methods for heart stimulation employing a multi-electrode array in contact with the heart, comprising: delivering a first pulse to a first set of electrodes, the first set of electrodes comprising at least one cathodal electrode and at least one anodal electrode; delivering a second pulse to a second set of electrodes, the second set of electrodes comprising at least one cathodal electrode and at least one anodal electrode, wherein the first set of electrodes and the second set of electrodes comprise at least one electrode in common, the first and second pulses received by the at least one electrode in common being opposite phases, and wherein at least the first pulse or the second pulse effects a pacing impulse between its at least one cathodal and at least one anodal electrodes.

Also disclosed are methods for heart stimulation, employing a multi-electrode array in contact with the heart, the multi-electrode array having at least three electrodes, comprising a) delivering a first pulse to the first electrode and a first pulse to the second electrode so as to effect a pacing impulse between the first and second electrodes, the first pulse to the first electrode being having a an opposite polarity opposite pulse than to the first pulse to the second electrode; and b) delivering a second pulse to the second electrode and a second pulse to the third electrode so as to effect a pacing impulse between the second and third electrodes, the second pulse to the second electrode being an opposite polarity pulse than the first pulse to the second electrode; the second pulse to the second electrode having a being an opposite polarity opposite pulse than to the first pulse to the second electrode.

Also provided are methods for sensing cardiac electrical activation, comprising: positioning a distal electrode of a right ventricle lead in the apex region of the right ventricle of a subject or to a right ventricular septum; positioning a proximal electrode of the right ventricular lead near a basilar portion of the right ventricle; employing the electrodes of the right ventricle lead to collect electrical signals generated by the right ventricle; positioning a first electrode of a left ventricular lead in a first branch of the coronary sinus of a left ventricle; positioning a second electrode of the left ventricle lead in a second branch of the coronary sinus of the left ventricle; and employing the electrodes of the left ventricular lead to collect electrical signals generated by the left ventricle.

The present disclosure also provides methods for cardiac resynchronization therapy, comprising positioning a distal electrode of a right ventricle lead in the region of the apex of a right ventricle of a subject or distal to a right ventricular septum; positioning a proximal electrode of the right ventricular lead in the region of the in the region of the basilar portion of the right ventricle; positioning a first electrode of a left ventricular lead in a first branch of the coronary sinus of a left ventricle; positioning a second electrode of the left ventricle lead in a second branch of the coronary sinus of the left ventricle; wherein the electrodes are electrically connected with a pulse generator by a lead comprising a conductor and that conductor electrically is isolated by insulation; delivering a first pulse to a first set of electrodes, the first set comprising at least one cathodal electrode and at least one anodal electrode; delivering a second pulse to a second set of electrodes, the second set of electrodes comprising at least one cathodal electrode and at least one anodal electrode, wherein the first set of electrodes and the second set of electrodes comprise overlapping electrodes having at least one electrode in common, the first and second pulses received by the at least one electrode in common being opposite phases, and wherein at least the first pulse or the second pulse effects a pacing impulse between its at least one cathodal and at least one anodal electrodes.

Further disclosed are methods for determining a pulse sequence of a multi-electrode array comprising determining a unipolar capture threshold for each electrode in the array; determining an electrode pacing order, wherein the electrode pacing order is in ascending order of the unipolar capture thresholds of the electrodes; determining a threshold for a pacing orientation for each pulse delivered to the array, wherein each pulse is delivered to a first electrode and a next electrode in the electrode pacing order; designing a pulse sequence for the array; and applying the pulse sequence to the array.

Also disclosed herein are methods of determining an overlapping multiphasic stimulation pulsing sequence in a multi-electrode array comprising selecting a pulse unit; assessing unipolar capture threshold for each electrode in the array for a given pulse width, orientation, and number of pulses; and designing a pulse sequence that requires a minimum number of pulses; and applying the pulse sequence to a cardiac resynchronization system.

Pacing systems are provided comprising an adaptor comprising electrically connecting electrodes disposed on three separate pacing leads to a single electrical connection with four electrodes.

### BRIEF DESCRIPTION OF THE DRAWINGS

The summary, as well as the following detailed description, is further understood when read in conjunction with the appended drawings. For the purpose of illustrating the disclosed methods and devices, there are shown in the drawings exemplary embodiments of the methods and devices; however, the methods and devices are not limited to the specific embodiments disclosed. In the drawings:

FIGS. 1A and 1B illustrate embodiments of the present invention directed to a four electrode pacing configuration;

FIG. 2 illustrates embodiments of right ventricular pacing;

FIGS. 3A and 3B illustrate alternative embodiments of a right ventricular pacing lead and defibrillation leads;

FIG. 4 illustrates an embodiment of an outer left ventricular lead;

FIG. 5 illustrates an embodiment of the proximal portion of the left ventricular lead system;

FIG. 6 illustrates an embodiment of the distal portion of the left ventricular lead system;

FIGS. 7A and 7B illustrate electrode configurations of the present invention;

FIGS. 8A and 8B illustrate electrode configurations of the present invention;

FIGS. 9A and 9B illustrate electrode configurations of the present invention;

FIG. 10 illustrates embodiments of electrode stimulation of the present invention;

FIG. 11 illustrates embodiments of electrode stimulation of the present; invention.

FIG. 12 illustrates embodiments of the right ventricular pacing lead and defibrillation leads;

FIG. 13 depicts steps for designing a sequential overlapping biphasic stimulation pulse sequence with autocapture and optimization;

FIG. 14 depicts steps for designing a sequential overlapping biphasic stimulation pulse sequence for pairs of electrodes;

FIG. 15 depicts steps for designing an overlapping multiphasic stimulation pulse sequence with autocapture and optimization;

FIG. 16 depicts steps for designing an overlapping multiphasic stimulation pulse sequence;

FIG. 17 depicts overlapping multiphasic stimulation optimized pulse sequence.

FIG. 18 illustrates an exemplary configuration for pacing in an approximately tetrahedral configuration around the heart with a conventional ICD;

FIG. 19 depicts an adaptor capable of electrically connecting three separate leads to an IS-4 connector; and

FIG. 20 depicts an alternative embodiment of an adaptor capable of electrically connecting three separate leads to an IS-4 connector.

### DETAILED DESCRIPTION OF ILLUSTRATIVE EMBODIMENTS

The disclosed methods and devices may be understood more readily by reference to the following detailed description taken in connection with the accompanying figures, which form a part of this disclosure. It is to be understood that the disclosed methods and devices are not limited to the specific methods and devices described and/or shown herein, and that the terminology used herein is for the purpose of describing particular embodiments by way of example only and is not intended to be limiting of the claimed methods and devices.

Unless specifically stated otherwise, any description as to a possible mechanism or mode of action or reason for improvement is meant to be illustrative only, and the disclosed methods and devices are not to be constrained by the correctness or incorrectness of any such suggested mechanism or mode of action or reason for improvement.

Throughout this text, the descriptions refer to compositions and methods of using said compositions. Where the disclosure describes or claims a feature or embodiment associated with a composition, such a feature or embodiment is equally applicable to the methods of using said composition. Likewise, where the disclosure describes or claims a feature or embodiment associated with a method of using a composition, such a feature or embodiment is equally applicable to the composition.

When a range of values is expressed, another embodiment includes from the one particular value and/or to the other particular value. Further, reference to values stated in ranges include each and every value within that range. All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods and devices which are, for clarity, described herein in the context of separate embodiments, may also be provided in combination in a single embodiment. Conversely, various features of the disclosed methods and devices that are, for brevity, described in the context of a single embodiment, may also be provided separately or in any subcombination.

The term “about” when used in reference to numerical ranges, cutoffs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

All standards referred to herein are the standards in place as of the day of filing of this application. For example, references to standards for connectors or leads, such as IS-1 and IS-4, are the standards in effect as of the filing date of this application.

As used herein, the singular forms “a,” “an,” and “the” include the plural.

As used herein, “pacing impulse” or “pacing pulse” can be used interchangeably and refer to the delivery of electrical current directly to the heart using a cathode electrode, an anode electrode, or both for a defined duration and voltage. Typical voltages for pacing are 1V-4V, and typical duration of pacing or pulse width is 0.1 ms to 1.0 ms.

As used herein, X-“point pacing” refers to pacing the heart using X number of electrodes. For example, in four point pacing, four electrodes are used to pace the heart. CRT systems can be referred to as “X-point CRT systems” or “X-point CRT.” For example, a four point CRT system employs four electrodes to pace the heart. Stated another way, a four point CRT system employs four point pacing.

Various terms relating to aspects of the description are used throughout the specification and claims. Such terms are to be given their ordinary meaning in the art unless otherwise indicated. Other specifically defined terms are to be construed in a manner consistent with the definitions provided herein. The term “comprise” includes its standard, open-ended meaning, but should also be understood as including “consist.” For example, an embodiment that comprises first and second components may include those components as well as additional parts, but may also include those two components and nothing more.

The most commonly used clinical approach for cardiac resynchronization therapy (CRT) involves transvenous placement of a right ventricular (RV) lead, with its tip positioned in the right ventricle, and placement of a coronary sinus or left ventricular (LV) lead with its tip positioned in the lateral branch of the coronary sinus. A lateral branch of the coronary sinus correlates to the lateral wall of the left ventricle. The leads have multiple distal electrodes for pacing the heart. The two leads are connected to a pulse generator typically implanted in a left pre-pectoral position which then provides pacing pulses to electrodes on the leads in a sequential manner. The term “pulse generator” is used herein as a generic term for either an implantable cardiac pacemaker or for an implantable cardioverter-defibrillator (ICD). “Leads,” as used herein, may refer to either pacing or ICD leads.

A second approach to cardiac resynchronization therapy is fusion pacing. This involves placement of the same leads in the heart and connecting those leads to a pulse generator. Fusion pacing may be performed so that pacing is delivered to the coronary sinus lead in a manner that is timed to electrical activation of the right ventricle. This approach uses the local electrical activity on the RV lead to determine timing of pacing on the LV lead (e.g., U.S. Pat. No. 6,871,096).

A third approach to multisite pacing is pacing from two separate electrodes on one LV lead in combination with pacing from one site on a RV lead. This approach with three site pacing has been reported to improve ventricular function more than standard biventricular pacing (e.g., Heart Rhythm 2015; 12: 1250-1258).

A fourth approach to multisite pacing also involves three site pacing, wherein two separate LV leads are implanted and positioned in different branches of the coronary sinus. These two LV leads are then ‘Y’ connected so that the electrodes are electrically in parallel. Pacing is first delivered simultaneously to the two LV electrodes with one pulse from the pulse generator and then to the RV lead, the third site for resynchronization (e.g., J. Cardiovasc Electrophysiol, Vol. 23, pp. 1228-1236, November 2012).

The standard approach to pacing the heart is to deliver a positive pulse to one electrode (the cathode) relative to a second electrode (the anode). If the second electrode is close (generally less than about 2 cm, but can be less than 3 cm, 4 cm, or even 5 cm because, as one skilled in the art understands, heart anatomy may differ between subjects) to the first electrode this is termed “bipolar pacing.” If the second electrode is the pulse generator or a coil of an ICD shocking lead, then pacing is termed “unipolar pacing.” “Cathodal pacing” occurs when the cathode receives a pulse, and “anodal pacing” occurs when the anode receives a negative pulse. Typically, the anode pacing threshold is higher than the cathode pacing threshold, and so the anode does not usually result in electrical excitation of the heart when the pacing threshold of the cathode is within a normal range. If the pacing threshold of the cathode is unusually high, then the anode may have sufficient output for anodal capture resulting in electrical excitation of the heart.

An alternative to cathode pacing that has not been widely used is biphasic pacing. In this method, an initial negative pulse is delivered followed by a positive pulse. The total energy and total current drain on the pulse generator with biphasic pacing has been reported to similar or less than with cathode pacing (e.g., PACE, Vol 13, pp. 1268-1276, October 1990, Ann Noninvasive Electrocardiol 2011; 16(2):111-116). If the series of pulses is reversed such that an initial positive pulse is followed by a negative pulse, then the pacing threshold and total current drain is typically higher than pacing with an initial negative pulse followed by a negative pulse.

Monophasic cathodal stimulation is more efficient than monophasic anodal stimulation to initiate a wavefront of depolarization. But biphasic pacing with an initial anodal phase followed by a cathodal phase generally requires similar or less total energy than a monophasic cathodal pulse. Conversely, biphasic pacing with an initial cathodal phase followed by an anodal phase generally requires similar or more total energy than a monophasic cathodal pulse.

Monophasic cathodal stimulation results in depolarization of cardiac tissues directly under the electrode, and the depolarized area may be dog-bone shaped with greater depolarization along the length, compared to the width, of the associated myocardial fibers. The depolarized area of myocardium is referred to as a virtual cathode. Due to that same pacing stimulus, in adjacent areas to the virtual cathode, an area of myocardium is hyperpolarized and is referred to as a “virtual anodal electrode.” Monophasic anodal stimulation results in hyperpolarization of cardiac tissues directly under the electrode, and the hyperpolarized area may be dog-bone shaped with greater hyperpolarization along the length, compared to the width, of the associated myocardial fibers than across the width of the myocardial fibers. In adjacent areas, virtual cathodal electrodes are created by that same pacing impulse and if the anodal stimulus is sufficiently strong, the virtual cathodal electrodes may be sufficient to initiate a depolarization wavefront.

Given the complexity of myocardial structure, including the consequences of disease and aging, and distance of an electrode to viable myocardium it is difficult to predict a consistently optimal pacing configuration to generate a local cardiac impulse from an electrode. An anodal pulse followed by a cathodal pulse may be employed in some situations to reduce energy requirements. Other tissue may be optimally stimulated by a cathodal pulse followed by an anodal pulse. Areas with a higher pacing threshold may require a higher magnitude of the voltage applied, a greater duration of total pulse width, or a greater number of phase transitions between cathodal and anodal impulses. Each of these parameters may be modified to identify a local pacing threshold and pacing safety margin.

The ventricles of the heart electrically activate by sodium channel dependent depolarization currents. The depolarization time period of the heart can be measured on an electrocardiogram (EKG) and the ventricular depolarization time period is measured as the QRS duration. After electrical excitation through excitation contraction coupling there is mechanical activation of the heart. Pathologic conditions such as a left bundle branch block or heart failure result in intraventricular conduction delays and a wide QRS. The wide QRS or electrical dyssynchrony then produces mechanical dyssynchrony. One powerful predictor of response efficacy to multisite pacing and resynchronization therapy is the magnitude of decrease in QRS duration in response to multisite pacing therapy when compared to the QRS duration before multisite pacing is applied. Shortening of the QRS duration correlates to more synchronous electrical activation and thereby more synchronous mechanical activation. Improved electrical and mechanical activation correlates to greater improvements in ventricular function and heart failure status.

The present disclosure provides novel systems and methods for cardiac resynchronization therapy. The systems include novel leads for right and left ventricular pacing. The novel method described herein comprises overlapping multiphasic stimulation. Overlapping multiphasic stimulation reduces (or even minimizes) energy drain on the pulse generator while maximizing the number of electrodes stimulating the heart.

The CRT systems described herein comprises a right ventricular lead capable of having multi-site pacing such that the tip is paced in the region of the right ventricular apex or distal right ventricular septum and a proximal electrode is capable of pacing the basilar portion of the right ventricle. One embodiment of the present invention provides a multi-site right ventricular lead comprising a distal section having a distal fixation mechanism; and a proximal section having at least one electrode and an end adapted to be connected to a pulse generator. Some embodiments provide a distal section that is elongate along a central axis, the distal section having a terminus and having a distal fixation that extends away from the distal terminus; and a proximal section that is elongate along the central axis and in electrical communication with the distal section, the proximal section having at least one electrode and an end adapted to be connected to a pulse generator. In some aspects, the distal section further comprises a distal electrode. In some aspects, the multi-site right ventricular lead further comprises a distal coil.

In some embodiments of the present disclosure, the multisite right ventricular lead comprises a distal bipole, distal coil, and a proximal electrode. The proximal electrode is used for pacing the basilar portion of the right ventricle. In other aspects of the embodiment, the right ventricular lead comprises a distal integrated bipole with a distal active or passive fixation tip, a distal coil for defibrillation and two rings proximal to the distal coil for pacing the right ventricle.

As used herein, “fixation mechanism” can refer to an active fixation mechanism such as a distal helix or a passive fixation mechanism such as at least one prong. The fixation mechanism allows the lead to be safely attached to cardiac tissue. In some aspects, the fixation mechanism comprises a helical electrode.

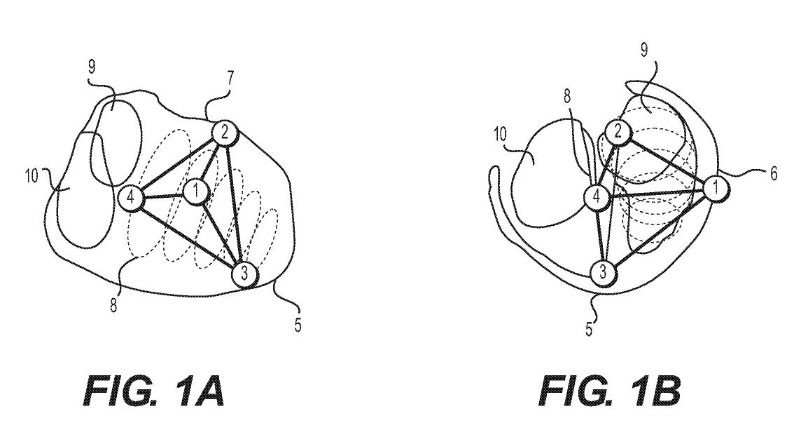

In one embodiment of the present invention, the first four point-pacing electrode configuration is as depicted in FIG. 1 with FIG. 1A being a right anterior oblique figure and FIG. 1B being a left anterior oblique figure. Regions of cardiac anatomy are labeled for orientation purposes including the right ventricular apex 5, the lateral left ventricle 6, than anterior left ventricle 7, the interventricular septum 8, the mitral valve 9, and the tricuspid valve 10. The multi-site RV pacing ICD lead is positioned in the right ventricular apex 5. The distal RV electrode 3, proximal RV electrode 4, the left posterior-lateral coronary sinus branch LV lead electrode 1, and the electrode of the anterior LV lead electrode 2 surround the left ventricle. Each pacing electrode may represent a vertex and this combination of vertices forms a tetrahedron. The linear distance between each of the vertices represents the edges of a tetrahedron. In one embodiment of the present invention, the aim at the time of implantation of the leads is to position the electrodes to approximate a tetrahedron around the left ventricle with similar lengths of the edges of the tetrahedron. Such a tetrahedral arrangement regularizes the distance between each electrode to all of the other electrodes. Also, the average distance between electrodes and left ventricular myocardium is reduced or even minimized and may create an improved resynchronization when compared to fewer pacing electrodes or alternative geometric configurations.

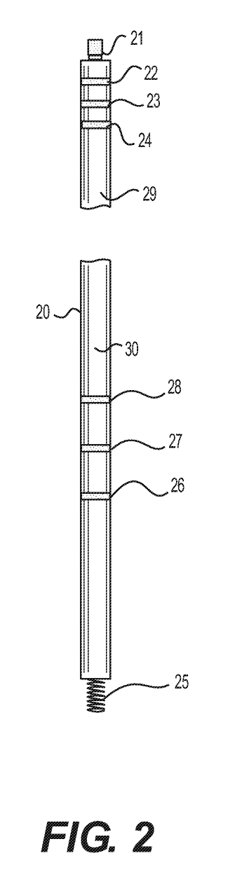

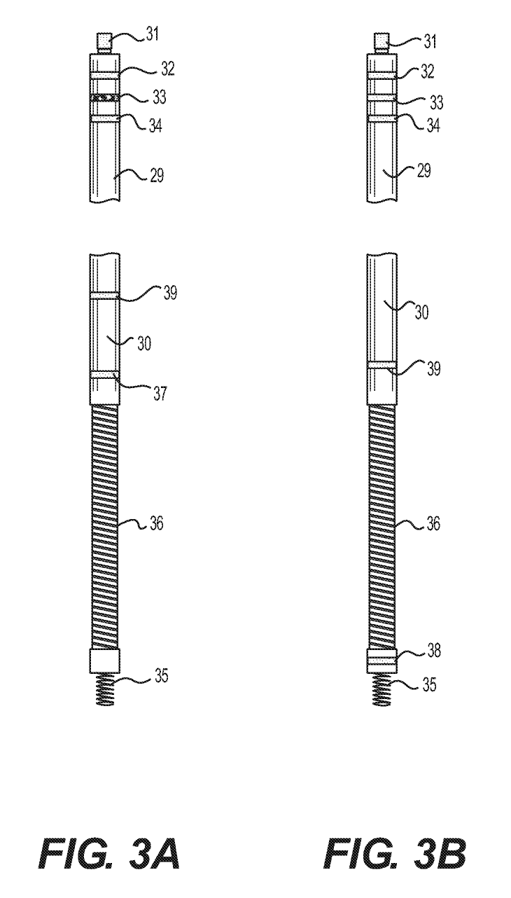

FIG. 2 illustrates one embodiment of a multi-site RV pacing lead 20 having three connecting ring electrodes 22, 23, 24 spaced to meet IS-4 standards and terminating in a connector 21 to electrically connect the lead to a pulse generator. In some embodiments, the connector is an IS-4 connector. Also, a multi-site RV pacing ICD lead may terminate in a connector such as an IS-4 connector. This type of connector may be used for a right ventricular quadrapolar pacing lead, an ICD lead with dedicated distal sensing with an ICD coil and a connecting ring electrode (FIG. 3A), or an ICD lead with integrated distal sensing with and ICD coil and two connecting ring electrodes (FIG. 3B). Other embodiments comprise IS-1 connectors or other connectors that are not recognized as industry standard connectors. FIG. 2 also illustrates the distal portion 30 of a pacing lead. This pacing lead may be a quadrapolar lead with a distal helix 25 used for attachment to myocardium. There is extended spacing between the distal helix electrode 25 and three electrode rings 26, 27, 28 (40 to 60 mm). There is spacing between the electrode rings 26, 27, 28 (10 mm to 20 mm). When the distal helix electrode 25 and one of the three electrodes 26, 27, 28 are selected for pacing, the proximal electrode 26, 27, 28 may be selected which has an optimal distance between electrodes and may accommodate large variations heart size.

Referring to FIG. 3A, a right ventricular ICD lead is shown. The embodiment illustrates an ICD lead with integrated distal sensing including a distal helix 35, which can be used for attachment and as an electrode, at the terminus of the distal portion 30 of the lead. The proximal end terminates in a connector 31 to electrically connect the lead to a pulse generator A distal coil 36 may be used for delivering an ICD shock, and in some aspects used for far-field sensing, and two ring electrodes 37, 39 may be used for pacing and sensing the basilar portion of the right ventricle. FIG. 3B illustrates an ICD lead with dedicated distal sensing including a distal helix 35, which can be used for attachment and as an electrode, at the terminus of the distal portion 30 of the lead, and a distal electrode ring 38 which may be used with the distal helix 35 for bipolar sensing. In some aspects, a distal coil 36 may be configured to for deliver an ICD shock, and the ring electrode 39 may be used for pacing and sensing the basilar portion of the right ventricle. The distance between the ring electrode 39 and the distal helix 35 allows for an improved distribution of electrodes for pacing. In terms of four point-pacing, each of those proximal and distal electrodes represents a vertex of a tetrahedron and the line connecting them an edge of the tetrahedron. It is desirable to have edges of the tetrahedron approximate equal edge lengths. However clinical factors such as patient anatomy may restrict locations of the vertices which result in a distorted tetrahedron. Generally, the more regular the edge lengths of the tetrahedron, the more even the spacing of the electrodes around the ventricle, and the synchronously the ventricle will activate. If the edge length vary by less than 50% then an adequate resynchronization should be achieved with the less the variation in edge length the better.

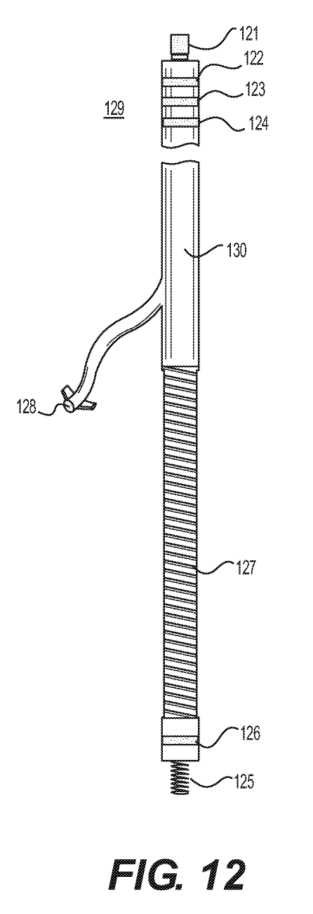

FIG. 12 depicts an embodiment of the right ventricular lead, wherein the lead has proximal portion 129 with connecting electrodes 121, 122, 123, and 124, a distal coil 127, a distal electrode 126, and a distal helix electrode 125, the distal helix being configured to attach the lead to cardiac tissue. The lead of FIG. 12 also comprises a branching electrode 128 with passive fixation tines to attach the lead to cardiac tissue. Proximal connecting electrodes 121, 122, 123, and 124 are electrically connected by an insulated conducting wire to electrode 125, 126, 127, and 128 respectively.

The herein disclosed technology also provides a left ventricular lead system. In some embodiments of the invention, this left ventricular lead system comprises an outer lead and an inner lead so that these lead tips may be deployed into separate branches of the coronary sinus and electrodes on the distal portion of these leads may deliver pacing to stimulate the heart with greater electrode separation than could be achieved with cannulation of a single branch of the coronary sinus. Furthermore, this lead system requires only one cannulation of the coronary sinus and only one lead body within the venous system outside of the coronary sinus. This lead is more easily deployable than prior attempts at dual branch pacing which required the use of two sheaths cannulating the coronary sinus. Furthermore, when used as an integrated system with overlapping multiphasic stimulation, substantial energy savings may be achieved over prior systems. Thus, the disclosures provide a left ventricular lead system, comprising an outer lead that is elongate along a central axis, the outer lead having a first end adapted to be connected to a pulse generator, a second end having at least one electrode; a first lumen that is disposed along or parallel to the central axis and between the first end and the second end of the outer lead and configured to receive a connector, the first lumen having a receiving port; and a second lumen configured to house a portion of the inner lead, the second lumen being disposed along or parallel to the central axis. The system also comprises an inner lead having a first end adapted to be connected to the receiving port, a second end having at least one electrode; and a securing mechanism disposed at or near the second lumen, the securing mechanism being configured to prevent the inner lead from moving in the second lumen once activated.

The proximal electrodes of the multi-site right ventricular pacing lead and the multi-site right ventricular ICD lead may be present in the form of a ring electrode. Alternative electrode designs are incorporated into the present invention as alternative embodiments. One alternative electrode design is to have the proximal electrode at the end an appendage or branch the main body of the lead. This branch includes a conductive wire, insulation, and a tip electrode. That tip electrode includes a fixation device such as a tine or an extendible helix.

One embodiment of the present invention provides a left ventricular lead system comprising an outer lead having a first end adapted to be connected to a pulse generator, a second end having at least one electrode, a first lumen having a port, and a second lumen; an inner lead having a first end adapted to be connected to the port, a second end having at least one electrode; a securing mechanism that once activated prevents the inner lead from moving through the second lumen. Preventing the inner lead from moving through the second lumen reduces the likelihood of the lead becoming displaced from the cardiac tissue.

Four point CRT systems as described herein utilize separate electrodes distributed around the left ventricle so that pacing from all of the electrodes results in a reduction or even minimization of the activation time for the entire left ventricle. Additionally, some embodiments of four point-CRT use overlapping multiphasic stimulation to simultaneously activate the heart with a reduced or even minimal energy requirement to activate all of the selected electrodes and generate the most synchronous activation by reducing or even minimizing electrical activation times to the entire left ventricle.

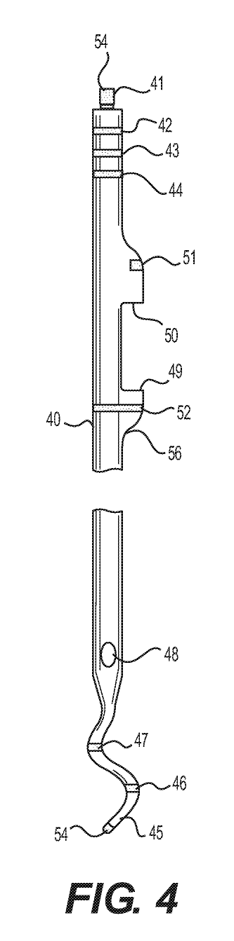

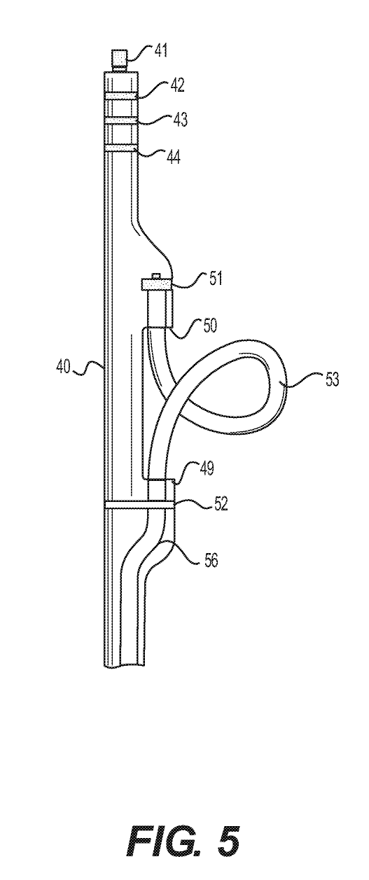

FIG. 4 illustrates an outer left ventricular lead 40. The proximal end 55 of the lead 40 terminates with a connector 41 adapted to receive signals from a pulse generator or transmit signals to a pulse generator. In one embodiment of the present disclosure, the proximal connector 41 is an IS-4 connector. Distal electrodes 45, 46, and 47 are connected by an insulated conducting wire to the proximal connecting electrodes 42, 43, and 44 respectively. The fourth electrode of the proximal connecting electrodes 44 is electrically connected to the hex screw connector 51 associated with a first lumen 50 which is designed to connect the outer lead 40 to an inner lead (FIG. 5). A second lumen 56 which starts at the proximal port 49 and ends at port 48 is designed for deployment of the inner lead. A securing mechanism 52 secures the inner lead to prevent the lead from moving through the second lumen 56. A third lumen 54 is designed for accepting stylets and guidewires for deployment of the distal tip 45 in a selected branch of the coronary sinus. This lumen 54 extends from the central portion of the proximal electrode 41 to the distal electrode 45.

FIG. 5 illustrates the inner lead 53 connected, via an IS-1 connector 51, to the first lumen 50 of the outer lead. The inner lead 53 may also be within the second lumen 56 of the sheath of the outer lead 40. The placement of the electrode on the distal portion of the inner lead is accomplished by pushing more lead 53 through the second lumen 56 or withdrawing an amount of the inner lead 53 from the second lumen 56. Once properly positioned, the inner lead 53 is prevented from further movement by a securing mechanism 52. In some aspects, the securing mechanism 52 is a suture sleeve. FIG. 5 shows a suture sleeve 52, which surrounds the sheath of the outer lead 40 at the second lumen 56. Once tightened, the suture sleeve 52 will prevent the inner lead 53 from moving inward or withdrawing from the coronary sinus. The suture sleeve 52 is only one possible securing mechanism. Other securing mechanisms include, but are not limited to, ferrules and other crimping mechanisms. In some aspects of the present invention, excess inner lead 53 will be positioned between the first lumen 50 and second lumen 56.

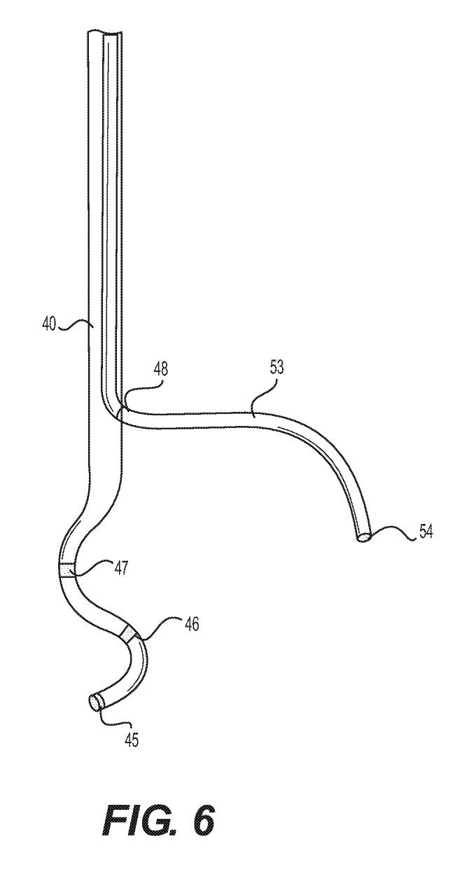

FIG. 6 illustrates the distal end of the left ventricular lead system. There is a side port 48 of the outer lead 40 that allows for advancement of the inner lead 53 through the outer lead 40 to an operator selected vessel. Electrodes 45 and 54 are positioned at or near the termini of the outer 40 and inner leads 53 and internal electrodes 46 and 47 are positioned proximal to the distal electrode 45.

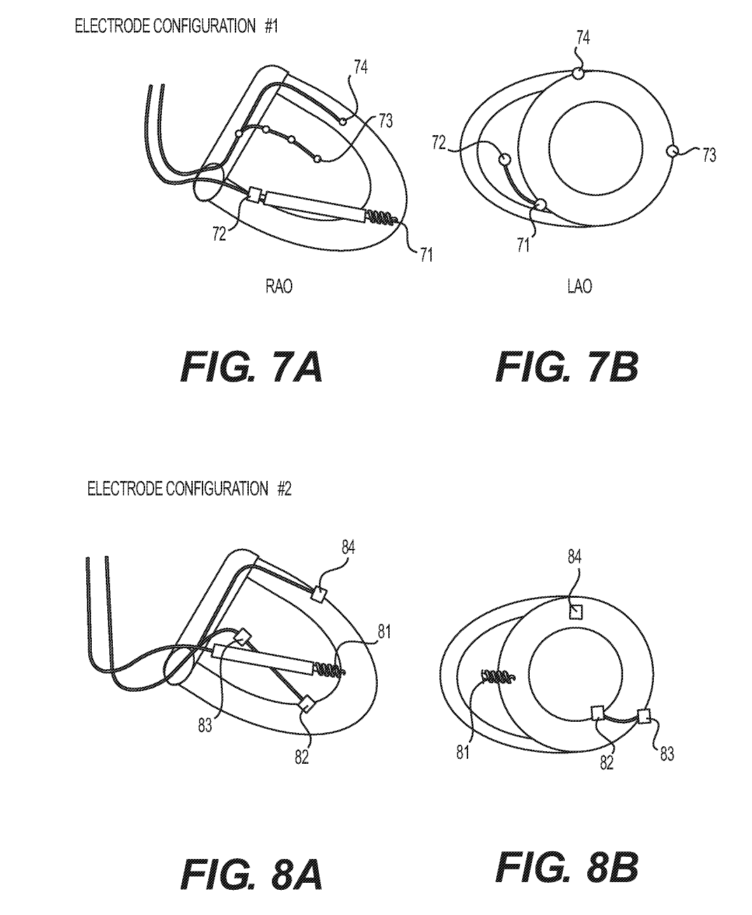

In some aspects of the present disclosure, the CRT system comprises four pacing sites from a number of potential electrode configurations. One configuration of the system is illustrated by FIG. 7A, which is a right anterior oblique view and FIG. 7B, which is a left anterior oblique view of the heart with a multi-site RV pacing ICD lead and the left ventricular lead system described above. In this configuration, a multi-site RV pacing ICD lead with the distal helix 71 is deployed in the right ventricular apex and proximal electrode 72 is positioned near the base of the right ventricle. The left ventricular lead system is implanted with the outer lead 73 in a lateral branch of the coronary sinus and the inner lead in an anterior branch of the coronary sinus. An electrode of the outer lead 73 is selected for pacing and the electrode 74 of the inner lead is also selected for pacing.

Another embodiment of the present disclosure is illustrated by FIG. 8A which is a right anterior oblique view and FIG. 8B which is a left anterior oblique view of the heart with an RV pacing ICD lead and the left ventricular lead system. In this configuration, an RV ICD lead with the distal helix 81 is deployed in the right ventricular septum. The left ventricular lead system is implanted with the outer lead in a lateral branch of the coronary sinus and the inner lead in an anterior branch of the coronary sinus. Electrode of the outer lead at position 82 and 83 are selected for pacing and the electrode 84 of the inner lead is also selected for pacing. The four electrodes 81-84 generate a tetrahedron around the left ventricle. This configuration is notable for utilizing two electrodes of the outer lead of the left ventricular lead system whereas the first configuration utilized two electrodes from the right ventricular lead for pacing.

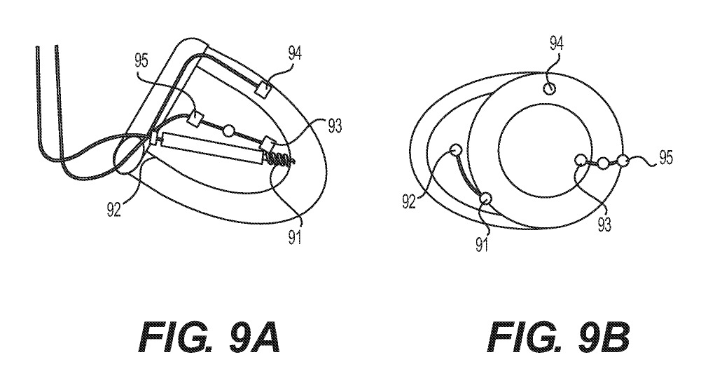

The present invention is not limited to four pacing sites and, as appropriate for a given patient's anatomy and heart failure status; additional embodiments include additional pacing sites. For example, the system can include five, six, or even more than six pacing sites. A third embodiment of the present disclosure is illustrated by FIG. 9A which is a right anterior oblique view and FIG. 9B which is a left anterior oblique view of a heart with an RV pacing ICD lead and the left ventricular lead system. In this configuration, five pacing sites are utilized for resynchronization. These are multi-site RV pacing 91 and 92 and three site LV pacing with two outer lead sites 93 and 95 and one inner lead site 94.

Some embodiments, four point-CRT provide that the a leadless pacemaker has an algorithm which uses an accelerometer to sense motion, rate smoothing, and to appropriately time pacing from the leadless pacemaker to the tetra-pacing system. If the coronary venous anatomy does not allow accommodation of the left ventricular lead system for optimal coverage of the left ventricle, there may be areas that remain late-activating and may benefit from pacing. The micro-pacemaker may be positioned in the left ventricular endocardium in an area that remains late-activating during four point-pacing. In some aspects, the micro-pacemaker could be electronically synchronized to pace at an appropriate time relative to pacing from the presently disclosed system to further improve synchrony of ventricular activation. Some aspects of the disclosure allow for placement of an independent microelectrode which interacts with the pacing system to improve resynchronization of the heart. Some embodiments provide a direct communication between the micro-pacemaker and the four point-pacing system. In some embodiments provide that the micro-pacemaker has an algorithm which uses an accelerometer to sense motion, rate smoothing, and to appropriately time pacing from the micro-pacemaker to the four point-pacing system.

For a given patient, an optimal electrode distribution is a set of electrodes that reduces or even minimizes the time for electrical wave fronts generated by the activation of the electrodes to fully excite (depolarize) the heart. If the number of electrodes for pacing the heart is four and the heart is approximated as a sphere with isotropic conduction, then a tetrahedron comprising equilateral triangles with the vertices of the tetrahedron on the surface of the sphere reduces or even minimizes the distance from the pacing sites to each of the sites on the sphere. Similarly, four point-pacing is pacing from four sites which surround the left ventricle to generate approximately equilateral triangles. Activation of myocardium occurs faster along the length of the myocytes than across the width of the myocytes (anisotropic conduction). The ventricle, although it becomes more spherical in dilated cardiomyopathy, it is not a sphere and has three-dimensional thickness, papillary muscles, and valves. Additionally, there may be unexcitable areas of the ventricle due to scarring. A tetrahedral orientation of electrodes provides an approximate orientation for an improved electrode distribution. However, additional electrode distributions may be considered which include pacing from a larger number of electrodes such as five, six, seven or even 8 electrodes. For these reasons, the systems of the present disclosure include a more effective anti-tachycardia pacing system since more areas of the heart will be activated more synchronously and is more capable of pacing into relative refractoriness than traditional cathodal stimulation.

The present disclosure also includes more advanced diagnostics for ventricular tachycardia capable of identifying the relative locations and sources of ventricular tachycardia. In some aspects of the present disclosure, data obtained from implanted leads is included in an advanced database of other episodes of ventricular tachycardia and ablation studies to guide ablation and antiarrhythmic drug approaches.

In some aspects of the present invention, the CRT system is configured as an implantable cardioverter-defibrillator or to have a permanent pacemaker. To ensure compatibility with existing standards, some configurations of the CRT system comprise an IS-1 connector for the atrial lead and two IS-4 connectors for the RV and LV leads. Each system includes a pulse generator typically implanted in the left pre-pectoral area. Three leads are implanted via venous access from the left shoulder area to reach the heart: one with its tip positioned in the right atrium, a second with its tip positioned in the right ventricle and a third with its two tips positioned in branches of the coronary sinus. Those skilled in the art will recognize that the pulse generator of the four point-ICD or four point-PPM system can be configured to include other standard types of leads including a standard single or dual-coil ICD lead with an IS-4 connector and/or a quadrapolar LV lead with an IS-4 connector. A skilled person would also recognize that the systems can be configured with non-standard connectors.

Some aspects of the leads disclosed herein provide for a quadrapolar lead with a single body, two lumens and a split to two separate branches, with each branch containing a lumen. In some aspects of the present disclosure, each branch contains a curvature or a fixation mechanism. In some aspects the fixation mechanism has one or more tines on the distal aspect of the lead. A “curvature” as used herein refers to a conformational change of the lead. Curvatures can facilitate implantation or provide passive fixation. Branches may be of different lengths. In some aspects, one branch is placed left lateral or posterior-lateral. In some aspects, the second branch is longer and placed anterior or anterio-lateral. In some aspects, each branch may include at least two electrodes. Since it is more common to have difficulty with phrenic nerve stimulation in the lateral position, an embodiment may have the lateral branch contain three electrodes and the anterior branch a single electrode. The four electrode selection is based on the current standards for hardware to contain electrodes. It should be understood that larger numbers of electrodes may be used, and the four-electrode embodiments discussed herein are illustrative only.

Another embodiment of the present disclosure provides for a left ventricular lead system comprising an electrode configuration having four electrodes on the distal aspect of both the inner and outer leads. In this configuration, the inner and outer leads may both have proximal IS-4 connectors so that all of the distal electrodes may be connected to a pulse generator. In this configuration, the outer lead would no longer contain the proximal IS-1 connector allowing for a comparatively large number of electrodes to pace the heart. When used in conjunction with a right ventricular lead, up to twelve electrodes would be available for pacing.

Also provided herein are methods for deploying the left ventricular lead system disclosed herein comprising the standard steps of cannulation of the coronary sinus and performance of a venogram. Next, the outer lead is deployed through the outer sheath and a lateral or posterior-lateral branch of the coronary sinus is selected. Standard tools such as a guide wire may be deployed through the lumen of the distal portion of the outer lead or subselection tools may be used to cannulate the target vessel. The outer lead is then deployed into the target vessel. Next, the inner lead is deployed through the outer lead with a guidewire. The guidewire is directed to an anterior interventricular vein or anterolateral branch of the coronary sinus. The inner lead is then advanced over the guidewire to the target vessel location. Now both the inner and outer leads are positioned in their target vessels.

Also provided are methods for securing the lead positions. In some aspects of these methods, the outer sheath is removed with standard sheath splitting tools. Bleeding at the site of vascular access is controlled with a suture. A suture sleeve around the outer lead near the vascular access site is used to secure the lead to the pectoral muscle or pre-pectoral fascia. The inner lead is secured relative to the outer lead with a second suture sleeve. In some aspects, the suture sleeve is built into the left ventricular lead system. The inner lead may be connected to the proximal portion of the outer lead via an IS-1 connector. Now, the distal electrodes of the inner and outer leads are electrically connected to the most proximal portion of the outer lead which is an IS-4 connector. The IS-4 connector may now be attached to the pulse generator. Those skilled in the art will recognize that variations in the implantation technique may involve additional steps to facilitate delivery of the outer lead or inner lead or removing steps when circumstances dictate that they are not necessary for deployment of the leads. The general process is engagement of the coronary sinus, assessment of vascular anatomy, deployment of the outer and inner leads, and securing the leads so that they do not become dislodged.

Provided herein are methods and devices for overlapping multiphasic stimulation, a novel cardiac pacing method that allows for development of a customized pulse sequence to deliver pacing pulses to all of the electrodes in rapid succession. Each electrode receives a set of pulses with changing orientation, or phase, from cathodal pulses to anodal pulses. Specifying the polarity of a pulse as cathodal or anodal defines the phase of the pulse. When the polarity of the pulse changes from cathodal to anodal, then there is a phase change. “Multiphasic,” as used herein, refers to a series of one or more phase changes between cathodal and anodal pulses delivered sequentially to an electrode. When desired, more than one cathodal or anodal pulse may be delivered in succession to the same electrode. The sum of pulses that an individual electrode receives may be monophasic, biphasic, triphasic, quadraphasic, or an even more complex combination of pulses.

The most basic form of overlapping multiphasic stimulation comprises utilizing two electrodes and delivers a biphasic pulse. In a bipolar pacing configuration, a first anodal pulse may be delivered to a first electrode which is paired with a first cathodal pulse delivered to a second electrode. The phase is reversed for the next pulse so that a second cathodal pulse may be delivered to the first electrode while the paired second electrode receives a second anodal pulse. Additional pulses may be delivered so that a triphasic, quadraphasic, or more pulses is delivered to each electrode.

Overlapping multiphasic stimulation is a type of ‘pulse sequence’ consisting of a series of electrical pulses delivered between a number of selected electrodes and/or a pulse generator. Each electrode will receive one or more pulses, those pulses may be paired to one or more different electrodes or pulse generator, and may be in an anodal or cathodal configuration. The goal of the pulse sequence is for each selected electrode to initiate a local cardiac depolarization wavefront. Each pulse of the pulse sequence consists of an electrical pulse delivered between two or more electrodes in a bipolar configuration. A pulse may also be between an electrode and the pulse generator or an ICD coil. Each pulse of the pulse sequence is characterized by a pulse sequence number, a cathodal electrode, an anodal electrode, a pulse magnitude, and a pulse width. Thus, each electrode in the pulse sequence will receive one or more pulses in a cathodal and/or anodal configuration as part of the pulse sequence.

“Overlapping” refers to pulses wherein one electrode receives a cathodal impulse while a second electrode receives an anodal impulse. Multiphasic refers to a single electrode that can receive a series of anodal and cathodal impulses. Series of pulses may oscillate between being an anodal and a cathodal configuration resulting in sequential phase changes, and each electrode may receive pulses paired in series with different electrodes.

The pulse sequence refers to the totality of pulses delivered between all of the electrodes. The electrode pulse sequence refers to the pulses received by a specific electrode which is a subset of the total pulse sequence. The local cardiac excitation threshold is a function of the locally delivered electrode pulse sequence's pulse width, pulse voltage, pulse orientation (anodal or cathodal), and number of phase transitions. Methods are therefore provided for method of heart stimulation employing a multi-electrode array in contact with the heart, comprising delivering a first pulse to a first set of electrodes in electrical communication with heart tissue, the first set of electrodes comprising at least one cathodal electrode and at least one anodal electrode; delivering a second pulse to a second set of electrodes, the second set of electrodes comprising at least one cathodal electrode and at least one anodal electrode, wherein the first set of electrodes and the second set of electrodes comprise overlapping electrodes having at least one electrode in common, the first and second pulses received by the at least one electrode in common being opposite phases, and wherein at least the first pulse or the second pulse effects a pacing pulse between its at least one cathodal and at least one anodal electrodes. In some aspects, the multi-electrode array comprises two electrodes. Some aspects provide for delivering additional pulses to the two electrode array, wherein each electrode receives an opposite phase pulse compared to an immediately previous pulse. These methods may also comprise delivering pulses to additional sets of electrodes, each additional set comprising at least one cathodal electrode and at least one anodal electrode, wherein each additional set of electrodes comprises at least one overlapping electrode in common from a set of electrodes receiving an immediately previous pulse. And in some aspects, pulses are delivered in a sequence such that each pulse is delivered to a unique electrode.

Because the methods above limit current drain, some aspects provide for the pulses to be delivered in a sequence that reduces or even minimizes energy expenditure from a power source.

In still other aspects of the methods described herein, the multi-electrode array comprises four electrodes, and in some aspects these electrodes may be arranged around, and in contact with, the heart in a tetrahedral fashion. Thus, in four point-CRT, the electrodes may be arranged in an approximately tetrahedral fashion. One skilled in the art will know that variations from the tetrahedral pattern may be necessary to accommodate a patient's anatomy. Such deviated arrangements of electrodes will be placed as nearly tetrahedrally as possible, and for the purposes of these disclosures such arrangements will be considered tetrahedral. For multi-electrode arrays comprising more than four electrodes, the more than four electrodes are arranged approximately equidistantly around, and in contact with, the heart.

Other methods disclosed herein provide method of heart stimulation, employing a multi-electrode array in contact with the heart, the multi-electrode array having at least three electrodes, comprising: a) delivering a first pulse to the first electrode and a first pulse to the second electrode so as to effect a pacing impulse between the first and second electrodes, the first pulse to the first electrode having a polarity opposite to the first pulse to the second electrode; and b) delivering a second pulse to the second electrode and a second pulse to the third electrode so as to effect a pacing impulse between the second and third electrodes, the second pulse to the second electrode having a polarity opposite to the first pulse to the second electrode. In some aspects, the array comprises more than three electrodes and wherein steps a) and b) are performed sequentially with the more than three electrodes. The steps of the method may be performed iteratively, and in some embodiments, the first pulse delivered to the first electrode is a depolarizing cathodal pulse.

One embodiment of the present disclosure provides a method for determining an overlapping multiphasic stimulation pulsing sequence in a multielectrode array comprising selecting a pulse unit; assessing unipolar capture threshold for each electrode in the array for a given pulse width, orientation, and number of pulses; designing a pulse sequence that requires a reduced or even minimum number of pulses, and applying the pulse sequence to the array.

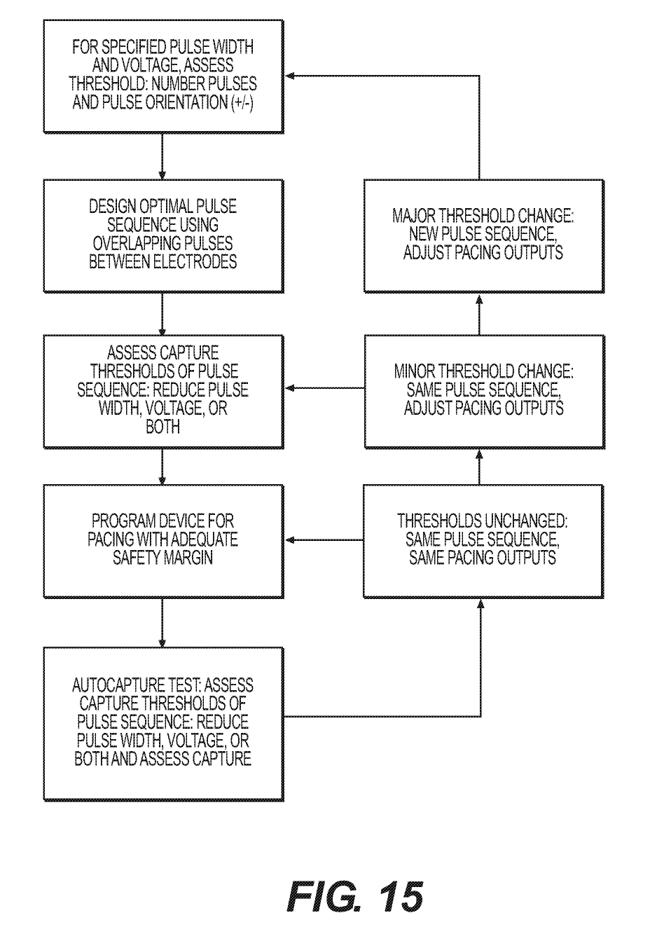

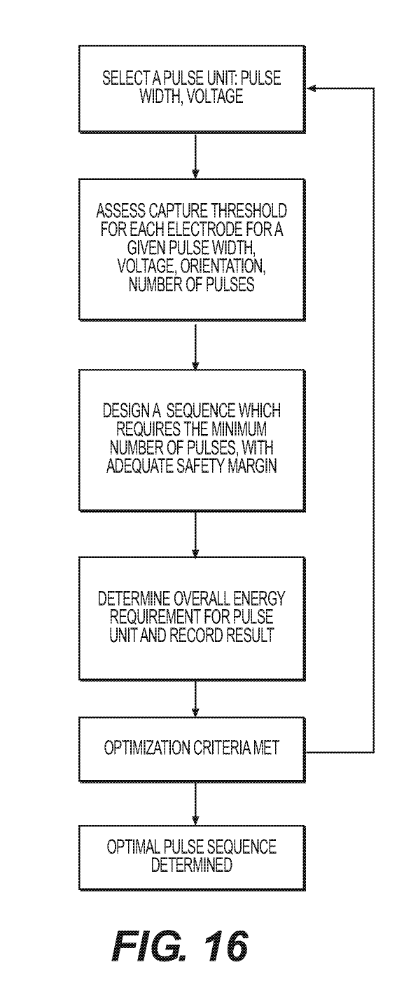

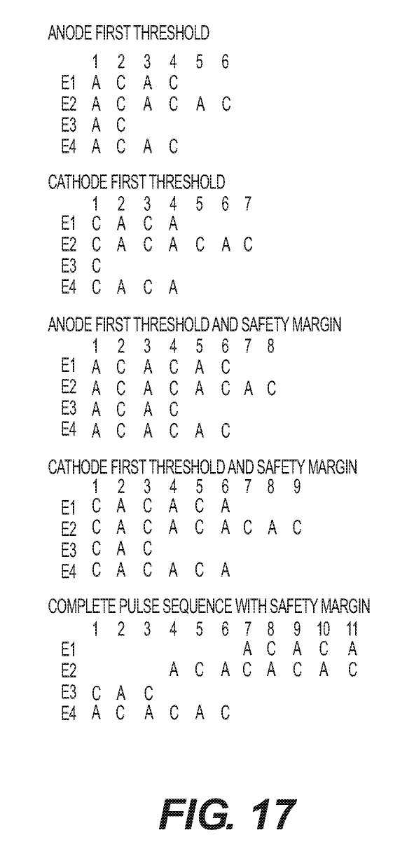

The process for designing a pulse sequence for overlapping multiphasic stimulation may be understood with reference to FIGS. 15-17. In this process, a pulse unit is selected that comprises a pulse width and a pulse amplitude. A capture threshold is determined for each electrode consisting of a series of pulses with that basic pulse unit. After capture thresholds are determined, a pulse sequence may be designed to deliver adequate pulses to each electrode to assure an adequate pacing threshold safety margin. The overall energy requirement for that pulse sequence may be measured. Alternative pulse units with different pulse widths and or pulse amplitudes may be assessed, and for the pulse unit, the total energy requirement for adequate pacing from each electrode may be measured. An optimal pulse unit and pulse sequence may then be determined to reduce or even minimize energy requirements and assure adequate capture from all electrodes.

In some aspects of the present disclosure, algorithms may be applied to identify electrode pulse sequences that successfully result in local depolarization of cardiac tissue by first referencing an electrode to the pulse generator and then identifying the local capture threshold. This may be performed at all of the electrodes of the total pulse sequence. If each pulse is at a specified amplitude and pulse width, then each electrode pulse sequence includes a uniform set of pulses. Typically, the orientation of cathode and anode will switch with each new pulse in an electrode pulse sequence. Once the electrode pulse sequence is determined for each of the electrodes, then the total pulse sequence may be determined by overlapping electrode pulse sequence so that all of the electrodes receive their determined electrode pulse sequence while reducing or even minimizing pulses that are not paired with another electrode. Thus, a maximal amount of energy is usefully applied to initiate depolarization wavefronts from the electrodes. The total energy requirement may then be determined for the pulse sequence for a given pulse magnitude and pulse width.

The basic unit of the pulse sequence in this optimization approach is a pulse having a pulse magnitude and a pulse width. The optimization process may be repeated with a different pulse magnitude and a different pulse width and the total energy requirement for excitation from all of the electrodes may again be determined. This process may be repeated within a range of voltages, e.g., 1.0 V, 1.5 V, 2.0 V, 2.5 V, 3.0 V, and a range of pulse widths, e.g., 0.1 ms, 0.2 ms, 0.3 ms, 0.4 ms, and 0.5 ms. An individualized overall optimal pulse sequence may then be determined to result in initiation of an electrical wavefront from all of the electrodes with an overall reduced or even minimal energy requirement.

Those of skill of the art will recognize that the basic pulse unit need not be uniform for a pulse sequence. Indeed, there may be situations where a single electrode has a much higher pacing threshold than all other electrodes. The high threshold electrode may be paired with two of the other electrodes as part of the pulse sequence and a forth electrode may be paced at a lower voltage if it is advantageous for reducing or even minimizing total energy consumption. In general, use of multiple pulse widths and/or multiple voltages within a single pulse sequence may make optimization of the pulse sequence more difficult because it may be more difficult to overlap electrode pulse sequences such that total energy is reduced or even minimized of the total pulse sequence.

Also described herein is sequential overlapping biphasic stimulation, a type of overlapping multiphasic stimulation. Generally, this method includes a first electrode receiving a first pulse in an anodal configuration and associated with a second electrode receiving a cathodal pulse and a second pulse in a cathodal configuration associated with a third electrode receiving an anodal pulse. Thus the pulses are sequentially delivered in an overlapping manner so that each electrode receives biphasic stimulation.

Sequential overlapping biphasic stimulation is a novel method of cardiac pacing using preconditioning anodal stimulation followed by depolarizing cathodal stimulation. The preconditioning anodal stimulation results in local hyperpolarization which allows for recruitment of additional sodium channels. Having additional sodium channels available, the magnitude of the cathodal stimulation required to initiate a depolarizing wavefront is less than without the preconditioning anodal stimulation.

One embodiment of the present disclosure provides methods for determining a pulse sequence and applying it to a multi-electrode array comprising determining a unipolar capture threshold for each electrode in the array; determining an electrode pacing order, wherein the electrode pacing order is in ascending order of the unipolar capture thresholds of the electrodes; determining a threshold for a pacing orientation for each pulse delivered to the array, wherein each pulse is delivered to a first electrode and a next electrode in the electrode pacing order; designing a pulse sequence for the array; and applying the pulse sequence to the array described herein.

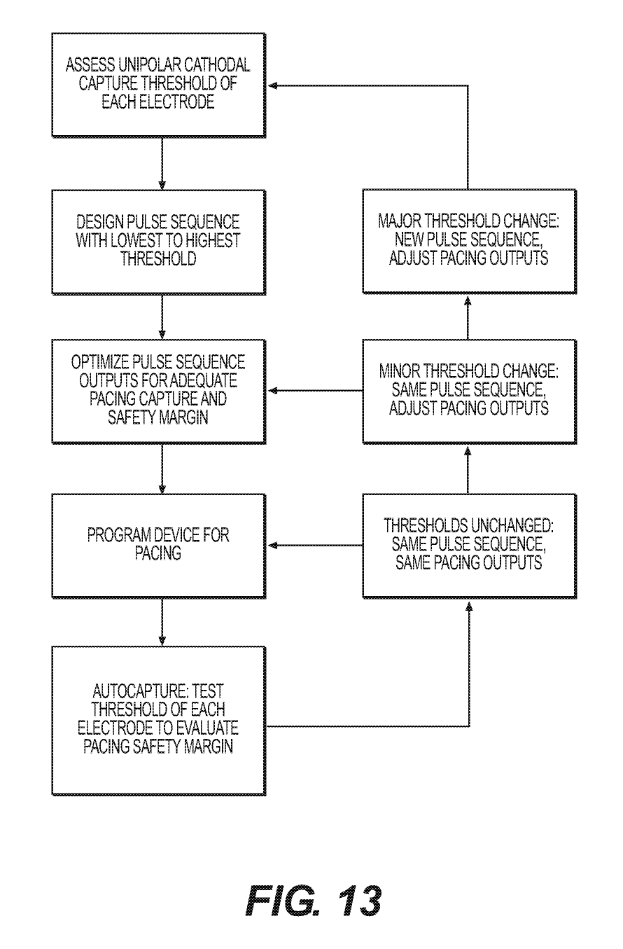

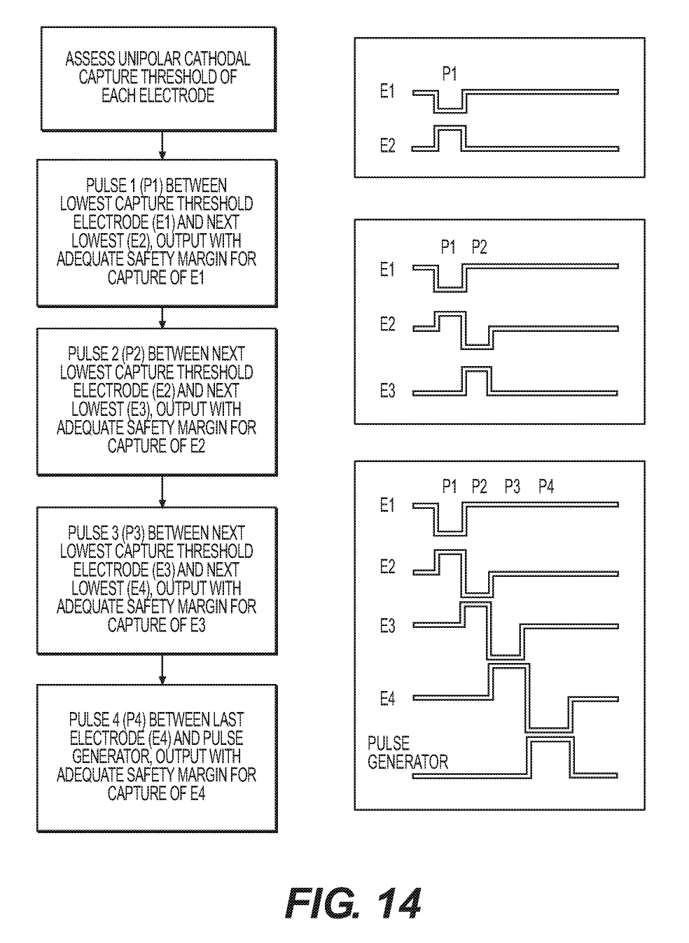

The process for designing a pulse sequence for sequential overlapping biphasic stimulation may be understood with reference to FIGS. 13 and 14. In some aspects of this process, the unipolar capture threshold for each electrode is assessed. This may be an automated process wherein each electrode is paced relative to an indifferent electrode which may be the pulse generator. Once each threshold is determined, then the electrode pacing order may be determined such that the lowest threshold electrode is paced first followed by electrodes having sequential increasing thresholds until all electrodes have been paced.

In some embodiments of the presently disclosed methods, the first pulse of the pulse sequence (P1) is delivered between a first electrode (E1) and a second electrode (E2) so that E1 receives a cathodal pulse (−) and E2 receives an anodal pulse (+). The threshold is determined for that pacing orientation which may be different than the initial unipolar capture threshold test. P1 is then determined so that there is an adequate pacing capture threshold safety margin for capture of E1.

The second pulse of the pulse sequence (P2) is delivered between a second electrode (E2) and a third electrode (E3) so that E2 receives a cathodal pulse (−) and E3 receives an anodal pulse (+). Overall, E2 receives biphasic stimulation with a first anodal pulse from P1 and a second cathodal pulse from P2 (+/−). The threshold is determined for that pacing orientation in which both pulses are delivered (P1 and P2). P1 has been determined from the prior step and P2 is varied in pulse width, pulse amplitude or both for determining the biphasic pacing threshold. P2 is then determined so that there is an adequate pacing capture threshold safety margin for capture of E2 when integrated with the initial pulse P1.

The third pulse of the pulse sequence (P3) is determined in the same manner as P2 and will be between the third electrode (E3) and the fourth electrode (E4). An adequate safety margin will be determined. Safety margins may be determined by analyzing several factors such as a patient's comfort level, a physician's familiarity with the device or manufacture, experience, the level of cardiopathy. For example, a physician who is unfamiliar with a particular device may want to include a greater safety margin than she would use with a more familiar device. The safety margin can be expressed as a percentage of energy delivered to an electrode or simply in volts.

The fourth pulse of the pulse sequence (P4) is delivered between a fourth electrode (E4) and the pulse generator so that E4 receives a cathodal pulse (−) and the pulse generator receives an anodal pulse (+). Overall, E4 receives biphasic stimulation with a first anodal pulse from P3 and a second cathodal pulse from P4 (+/−). The threshold is determined for that pacing orientation in which both pulses are delivered (P3 and P4). P3 has been determined from the prior step and P4 is varied in pulse width, pulse amplitude or both for determining the biphasic pacing threshold. P4 is then determined so that there is an adequate pacing capture threshold safety margin for capture of E4 when integrated with the initial pulse P3.

After determining a pulse sequence for sequential overlapping biphasic stimulation with an adequate safety margin for pacing all selected electrodes, the pulse generator can be programmed to deliver sequential overlapping biphasic stimulation.

Some embodiments of the present disclosure provide for reassessing the capture threshold to assure that adequate resynchronization therapy continues to be delivered. This process called “autocapture” may repeat the initial determination of the pulse sequence. In other aspects, local capture may be assessed at electrode. For a given pulse sequence P1, P2, P3, P4, the first pulse P1 of the pulse sequence may be reduced in pulse width, pulse amplitude, or both until local capture is lost on E1. The threshold may then be recorded for E1. The process may be repeated for P2, P3 and P4 to determine the thresholds for all the electrodes.

If the thresholds are unchanged, then the pulse generator need not be altered. If there is a minor change in the pacing threshold, then the pacing output may be adjusted to assure an adequate pacing threshold. If there is a major change in the pacing threshold, the entire pulse sequence may need to be re-determined.

Some aspects of the present invention provide cardiac pacing that utilizes a discharging capacitor to deliver an exponentially decaying voltage over time between a cathode and an anode. When the duration of the cardiac pacing pulse, or pulse width, is short relative to the decay rate of the charge on the capacitor, the cardiac pacing pulse voltage may be viewed as essentially constant. Some embodiments of the present cardiac pacing circuits include a plurality of capacitors that deliver a plurality of pacing pulses between electrodes. Using a plurality of capacitors, allows for delivery of a variety of pulse voltages. In other embodiments of the present disclosure, cardiac pacing comprises applying a constant current over a specified pulse duration.

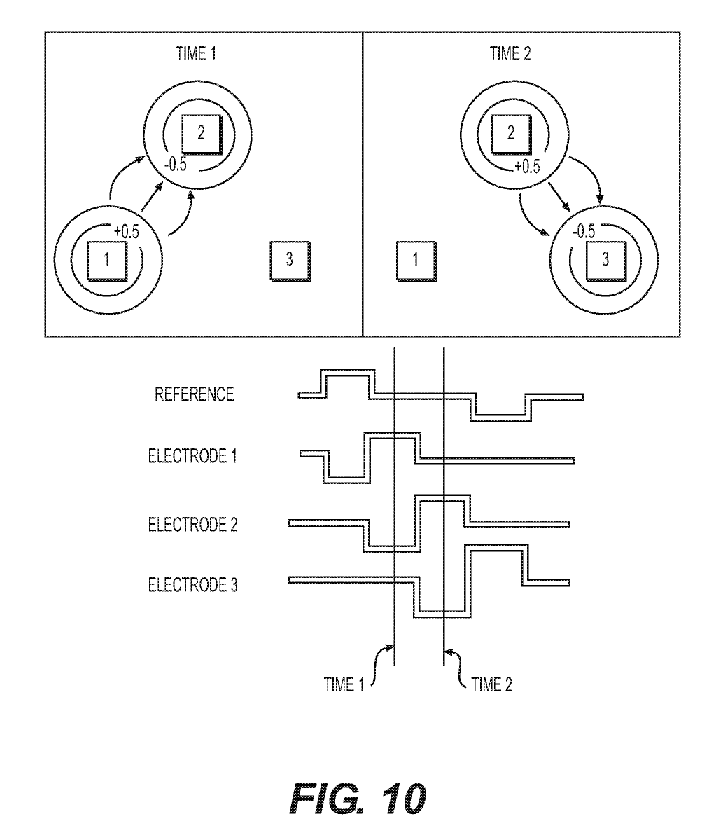

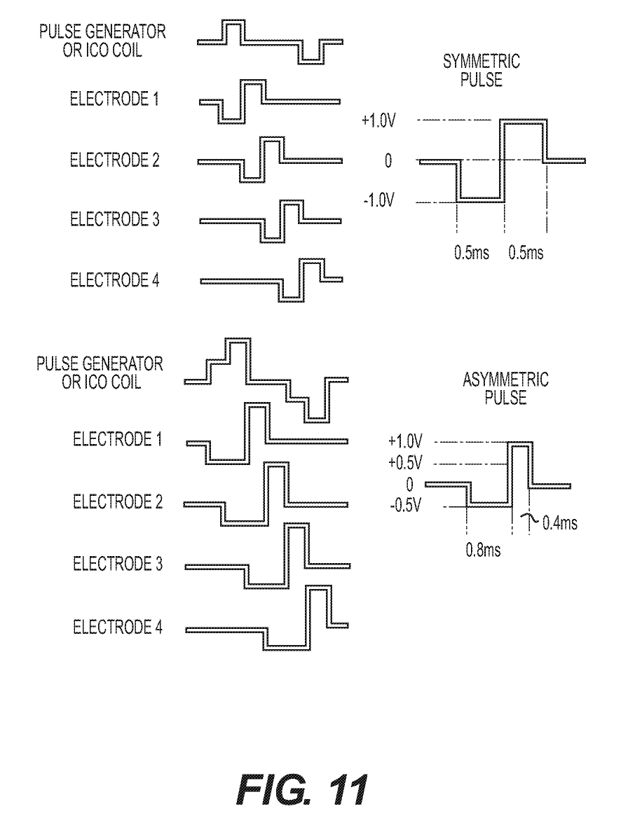

One embodiment, as exemplified in FIGS. 10 and 11, of the invention provides a method of heart stimulation employing a multi-electrode array in contact with the heart comprising delivering a first pulse to a first set of electrodes, the first set comprising at least one cathodal electrode and at least one anodal electrode; delivering a second pulse to a second set of electrodes, the second set comprising at least one cathodal electrode and at least one anodal electrode, wherein the first set of electrodes and the second set of electrodes comprise at least one electrode in common, the first and second pulses received by the at least one electrode in common being opposite phases, and wherein at least the first pulse or the second pulse effects a pacing impulse between its at least one cathodal and at least one anodal electrodes.

In some aspects, the multi-electrode array comprises two electrodes. Some embodiments of this method using two electrodes further comprise delivering additional pulses to the two electrode array, wherein each electrode receives an opposite phase pulse compared to an immediately previous pulse. Other aspects comprise delivering additional pulses to additional sets of electrodes, each additional set comprising at least one cathodal electrode and at least one anodal electrode, wherein each additional set comprises at least one electrode in common from a set of electrodes receiving an immediately previous pulse. Some pulses may be delivered, for example, to one cathodal electrode and two anodal electrodes.

Sequential pulses share an electrode in common (an “overlapping electrode”) in some embodiments, and in some embodiments the pulses are delivered in a sequence such that each pulse is delivered to a unique electrode. A “unique electrode” as used herein refers to an electrode that has not previously received a pulse in the sequence. In some embodiments, the overlapping electrodes of the array have an ascending order of thresholds and the pulses are delivered in a sequence according to the ascending order of thresholds. In some aspects, the pulses are delivered in a sequence that reduces or even minimizes energy expenditure from a power source.

Another embodiment of the present invention provides a method of heart stimulation employing a multi-electrode array in contact with the heart having at least three electrodes comprising a) delivering a first pulse to the first electrode and a first pulse to the second electrode to effect a pacing impulse between the first and second electrodes, the first pulse to the first electrode being an opposite polarity pulse than the first pulse to the second electrode; and b) delivering a second pulse to the second electrode and a second pulse to the third electrode to effect a pacing impulse between the second and third electrodes, the second pulse to the second electrode being an opposite polarity pulse than the first pulse to the second electrode. In one aspect, the array comprises more than three electrodes and steps a and b are performed sequentially with the additional electrodes. In another aspect, steps a and b are performed iteratively over all electrodes present in the array under the control of a preselected control program. In some embodiments, the first pulse delivered to the first electrode is a depolarizing cathodal pulse.

Another aspect of the present invention provides a method of heart stimulation employing a multi-electrode array having at least three electrodes comprising providing a pacing impulse between two electrodes by delivering a cathodal impulse to one electrode and an anodal impulse to the other electrode; and providing additional pacing impulses by delivering a cathodal impulse to an electrode that received an anodal impulse during a previous pacing impulse and delivering an anodal impulse to an electrode that received no impulse during the previous pacing impulse.

In a first example of sequential overlapping biphasic stimulation, a symmetric biphasic pulse is delivered to each electrode. As depicted in FIG. 2, electrode 2 receives a first pulse which is negative or anodal while electrode 1 receives a pulse which is opposite and positive or cathodal. The circuit involves pacing between electrode 1 and electrode 2. Next and with reduced or even minimal time delay electrode 2 receives a second, cathodal pulse while electrode 3 receives an anodal pulse. The next circuit in the sequence involves pacing between electrode 2 and electrode 3.

In one aspect of the present disclosure, an electrode receives a preconditioning anodal pulse to improve local excitability of cardiac tissue and is paired with a depolarizing cathodal pulse on a different electrode. While the electrode is receiving a depolarizing cathodal pulse, the next electrode may receive a preconditioning anodal pulse. Biphasic stimulation is hence performed using three electrodes for each electrode receiving a biphasic pulse. The voltage of each pulse and the duration of each pulse can be modified based on patient and electrode characteristics to assure local capture. Biphasic stimulation does not require that the anodal and cathodal pulses be symmetric. Each electrode may have different threshold characteristics for biphasic stimulation, such as anodal voltage and pulse width, and cathodal voltage and pulse width. When the pacing thresholds of each electrode are determined, then a pulse sequence can be designed and delivered to the leads.

While anodal stimulation from the coil of a right ventricular lead can improve resynchronization, there are currently no leads designed to exploit this behavior. Rather, contemporary methods and devices increasingly use single coil ICD leads, which leave an open electrode for use for resynchronization.

Multipoint resynchronization may be of additional benefit to patients; however, the distance between electrode positions of current quadrapolar leads and the RV lead may be insufficient. A solution presented herein increases the distance between electrodes on the LV quadrapolar lead; however, this distance may remain insufficient to efficiently capture a ventricle with great dispersion between electrodes.

The methods and devices described herein improve stimulation of the heart by reducing or even minimizing the total activation time of the heart, and in particular the left ventricle. Activation of the heart may occur at four locations: (1) the RV apex, (2) basilar portion of the RV, (3) left lateral branch of the CS lead, and (4) anterior coronary sinus. The goal of the overall configuration is to form a tetrahedron, i.e., equilateral triangles formed between each of the four pacing sites.

Pacing that results in the shortest total QRS duration and in particular the shortest total left ventricular activation time may have the greatest improvement in left ventricular mechanical synchrony. Sensing may be performed from each of the electrodes of the system relative to the generator. One embodiment of the present invention provides a method for sensing cardiac electrical activation comprising positioning a distal electrode of a right ventricle lead near an apex of a right ventricle of a subject or to a right ventricular septum; positioning a proximal electrode of the right ventricular lead near a basilar portion of the right ventricle; employing the electrodes of the right ventricle lead to collect electrical signals generated by the right ventricle; positioning a first electrode of a left ventricular lead in a first branch of the coronary sinus of a left ventricle; positioning a second electrode of the left ventricle lead in a second branch of the coronary sinus of the left ventricle; and employing the electrodes of the left ventricular lead to collect electrical signals generated by the left ventricle. In some aspects, this method for sensing cardiac electrical activation further comprises converting the collected electrical signals into electrograms. Furthermore, the method can also comprise using the electrograms and the subject's specific anatomy relative to the electrodes to model the subject's ventricular tachycardia. It is further contemplated in the present disclosure to store the electrical signals in a database. As this sensing method will detect irregularity in a subject's ventricular cardiac rhythm, in some aspects of the present invention, overlapping multiphasic stimulation is applied when the collected signals indicate a premature ventricular beat.

In some embodiments of the present invention, pacing is delivered monophasically. There may be a benefit of biphasic or multiphasic pacing to reduce total energy required to simulation the heart and or to facilitate stimulation of multiple poles simultaneously. Given the plurality of potential pacing configurations including waveforms (e.g., monophasic, biphasic, etc.) and vectors, an automated system can determine the appropriate pacing configuration to maximize battery longevity.

In one embodiment of the present invention, alternative pacing waveforms, including biphasic pacing, triphasic pacing, or quadraphasic pacing are used, which can reduce the energy required to depolarize local tissue. The use of alternative waveforms is expected to utilize 60-90% of the energy required to depolarize tissue using a standard monophasic cathodal waveform. When multiple electrodes are desired to be paced as described in the present disclosure, the use of overlap with other electrodes by pairing anodal and cathodal pulses may save an additional 50-70% in total energy when compared to pacing each site independently. This gives a total potential savings of 30-70%. Since there are many patient specific anatomic variables, the actual energy savings is difficult to predict but should be understood that by using multiphasic waveforms and overlap of those waveforms, a substantial energy savings may be achieved while activating cardiac tissue from a large number of electrodes.

One embodiment of the present invention provides a method of cardiac resynchronization therapy, comprising: positioning a distal electrode of a right ventricle lead near an apex of a right ventricle of a subject or distal to a right ventricular septum; positioning a proximal electrode of the right ventricular lead near a basilar portion of the right ventricle; positioning a first electrode of a left ventricular lead in a first branch of the coronary sinus of a left ventricle; positioning a second electrode of the left ventricle lead in a second branch of the coronary sinus of the left ventricle; wherein the electrodes are electrodes are electrically connected with a pulse generator by a lead comprising an electrically isolated conductor; and delivering a first pulse to a first set of electrodes, the first set comprising at least one cathodal electrode and at least one anodal electrode; delivering a second pulse to a second set of electrodes, the second set comprising at least one cathodal electrode and at least one anodal electrode, wherein the first set of electrodes and the second set of electrodes comprise at least one electrode in common, the first and second pulses received by the at least one electrode in common being opposite phases, and wherein at least the first pulse or the second pulse effects a pacing impulse between its at least one cathodal and at least one anodal electrodes. Insulating the conductor electrically isolates it from other components of the lead, as well as isolating it from other leads, other components of the system, and the patient. Some embodiments comprise delivering additional pulses to additional sets of electrodes, each additional set comprising at least one cathodal electrode and at least one anodal electrode, wherein each additional set comprises at least one electrode in common from a set of electrodes receiving an immediately previous pulse. Some pulses may be delivered, for example, to one cathodal electrode and two anodal electrodes. Sequential pulses share an electrode in common (an overlapping electrode) in some embodiments, and in some embodiments the pulses are delivered in a sequence such that each pulse is delivered to a unique electrode. In some embodiments, the overlapping electrodes of the array have an ascending order of thresholds and the pulses are delivered in a sequence according to the ascending order of thresholds. In some aspects of this embodiment, the pulses are delivered in a sequence that reduces or even minimizes energy expenditure from a power source. Some aspects of the method further comprise a micro-pacemaker synchronized to the pulse generator. In some aspects, the method further comprises delivering the pulses about simultaneously. In some aspects, the sequence of pulses is delivered such that each pair of electrodes receiving a pulse includes a first electrode that received an immediately previous pulse and a second electrode that did not receive the immediately previous pulse. In some aspects, the first electrode receiving a cathodal phase of a pulse received an anodal pulse during an immediately previous pulse.

Another embodiment of the present invention provides testing anodal stimulation from each of the electrodes. While monophasic cathodal stimulation, biphasic, triphasic, or quadraphasic stimulation are all generally more efficient than anodal stimulation of the heart to result in generation of a depolarizing wavefront, at times the closest packing structure for the total pulse sequence may result in an ‘unused’ anodal stimulus. If the threshold for anodal stimulation is sufficiently low and other pulse outputs are sufficiently high, then an anodal stimulus only may be used for pacing the heart for a specific electrode. This may improve the efficiency of the pulse sequence by compacting the pulse sequence into the least total energy required to pace the heart. Anodal stimulation threshold testing may be performed relative to the pulse generator from each electrode. Values of anodal stimulation may be recorded and/or integrated into a pulse sequence if it is found to conserve battery.

Sensing of the heart's electrical activity may be performed from one or more electrodes of the right ventricular and left ventricular leads. Sensing electrodes need not be restricted to the electrodes used for pacing. The sensed electrical activity from one or more of these electrodes may be processed with analog filtering and post-processed with rectification, wavelets, Hilbert transform, voltage derivative, or some combination of these techniques. A local activation time at one or more of the electrodes may be derived from one or more of these post-processed signals.

In one embodiment of the present disclosure, methods are disclosed for sensing cardiac function from four electrodes which are positioned in an approximate tetrahedron around the heart. In some embodiments, additional far field electrocardiograms using the pulse generator, the atrial lead, and/or ICD coils may be used to identify farfield electrical activity of the heart to approximate the surface electrocardiogram. An advantage of this approach (four sensing electrodes and electrocardiograms) is greater information regarding the activation sequence of the ventricle. When this information is combined with additional far field electrograms from the pulse generator and ICD coils, it is possible to identify the timing of activation of each of the electrodes relative to major depolarization wavefronts. In some embodiments, when combining the electrogram information with a CT scan of cardiac anatomy, patient specific modeling of the ventricular tachycardia is performed prior to an intended ablation procedure in patients with recurrent ventricular tachycardia.

For focal tachycardias, it is possible to triangulate the approximate location of a ventricular tachycardia source based on estimations of conduction times from source to each of four electrodes. For reentrant tachycardias, it is also possible to estimate the location of the ventricular tachycardia circuit based on relative timing of electrical activation on each of the four sensing electrodes, far field electrograms from Einthoven's triangle, and the patient's CT scan of cardiac anatomy and electrode positions.

Another embodiment of the present disclosure provides a method for sensing cardiac electrical activation comprising positioning a distal electrode of a right ventricle lead near an apex of a right ventricle or distal to a right ventricular septum; positioning a proximal electrode of the right ventricular lead near a basilar portion of the right ventricle; employing the electrodes of the right ventricle lead to collect electrical signals generated by the right ventricle; positioning a first electrode of a left ventricular lead in a first branch of the coronary sinus of a left ventricle; positioning a second electrode of the left ventricle lead in a second branch of the coronary sinus of the left ventricle; employing the electrodes of the left ventricular lead to collect electrical signals generated by the left ventricle; and converting the collected electrical signals into electrograms.

A database may be generated for patients who have a CRT-system as described herein. This database may include episodes of tachycardia, CT scans with electrode positions, cardiac dimensions and changes in cardiac dimensions, and response to pacing methods described herein. The database may also include results of electrophysiological study and ablation of ventricular tachycardia. The patient information contained in the database can be used in combination with patient specific anatomy relative to the electrodes for specific modeling of ventricular tachycardia. In one aspect, the database allows comparison between new episodes of tachycardia and prior recorded episodes of tachycardia and electrophysiological studies. Episodes of previous tachycardias, which have been targeted for ablation, can be predictive of the nature of new episodes of tachycardias documented on the CRT-system. This additional knowledge allows for greater accuracy in predicting the type of ventricular tachycardia and may assist in directing an ablation procedure.

In patients who have frequent ventricular premature beats or in patients with beats conducted aberrantly due to an atrial arrhythmia, e.g. atrial fibrillation, it is desirable to have the most synchronous contraction possible during the premature beat or aberrant beat. Some aspects of the disclosed activating sensing methods provide for applying overlapping multiphasic stimulation is applied to create a more uniform contraction than otherwise would have occurred with the ventricular premature beat or aberrant beat.

Ventricular tachyarrhythmic events such as monomorphic ventricular tachycardia, polymorphic ventricular tachycardia, ventricular flutter, and ventricular fibrillation are typically perpetuated by continuous depolarization wavefronts, referred to herein as “reentry paths”. These reentry paths may be stable and repeated as in the cases of monomorphic ventricular tachycardia and ventricular flutter, or these reentry paths may be unstable and not repeated continuously as in the cases of polymorphic ventricular tachycardia and ventricular fibrillation. Anti-tachycardia pacing has been developed for use in ICDs to terminate ventricular tachycardia by pacing the heart rapidly in an attempt to disrupt the continuous depolarization wavefront. In some aspects, efficacy of antitachycardia pacing is improved by pacing from multiple electrodes nearly simultaneously using overlapping multiphasic stimulation as described herein.

In some embodiments of the present invention, delivery of an anodal stimulus as part of biphasic pacing recruits additional sodium channels for excitation (PACE, Vol 13, October 1990; 1268-1276). Biphasic pacing results in enhanced cardiac excitability, i.e., the ability of pacing to result in capture of the heart (initiation of a new electrical wavefront) within the relative refractory period of cardiac tissue. Multiphasic pacing with triphasic or quadraphasic impulses may have similar impacts of being able to pace and result in capture of the heart within the relative refractory period. There are two mechanisms by which overlapping multiphasic stimulation (including sequential overlapping biphasic stimulation) is expected to be more effective than traditional single site, dual site, or triple site anti-tachycardia pacing. First, by having a greater spatial distribution of electrodes, the heart will be more simultaneously activated by the anti-tachycardia pacing and is expected to be more effective at disrupting a tachycardia circuit. Second, sequential overlapping biphasic stimulation is a pacing method that includes an anodal followed by a cathodal stimulus. The anodal stimulus component of biphasic pacing results in the ability to capture areas of the heart which would be refractory to cathodal stimulation alone. Thus, more electrodes will be successful in initiating wavefronts and creating more synchronous activation of the heart and disruption of the tachycardia mechanism. In some aspects of the present invention, anti-tachycardia pacing using sequential overlapping biphasic stimulation is more effective at terminating ventricular tachyarrhythmias by having greater penetration into arrhythmia circuit core.

Pacing sites which are chosen for CRT pacing as described herein are expected to be ideal for anti-tachycardia pacing. Some aspects provide for pacing from all available electrodes and the RV coil for presently described CRT ICD systems. Pacing from all electrodes using high-output sequential overlapping biphasic stimulation and including the RV coil is expected to directly activate an even greater number of myocardial sites and create the most synchronous potential activation possible from available leads. This results in even more effective anti-tachycardia pacing.

Patients with a previously implanted ICD system or biventricular ICD system may benefit from an upgrade to a CRT system as described herein. Depending on existing hardware, adaptation may include placement of a new LV lead and may require a Y-adapter at the proximal portion to attach IS-1 or IS-4 connector of the new LV lead and the IS-1 or IS-4 connector of the old LV lead into an IS-4 connector. Similarly, if additional right ventricular resynchronization is desired and there is an existing ICD lead or if the connecting rings of a new ICD lead are unable to pace the right ventricle, then a standard pacemaker lead with an IS-I connector may be implanted in the desired portion of the right ventricle. The ICD lead and the pacemaker lead implanted in the desired portion of the right ventricle may be connected to a Y-adapter which results in a single IS-4 connector which may be connected to a ICD pulse generator or a pacemaker pulse generator.

Other embodiments of this disclosure provide a system and methods for utilizing a conventional pulse generator (pacemaker or ICD), four conventional leads, and a specialized lead adaptor to perform pacing in a tetrahedral configuration around the left ventricle. In some aspects, the four conventional leads comprise an active or passive fixation, unipolar or bipolar pacing leads with an IS-1 connection; an active or passive fixation ICD leads with a DF-4 connection; a unipolar or bipolar LV lead with an IS-1 connection; and a bipolar or quadrapolar LV lead with an IS-1 or an IS-4 connection. The leads, with their electrodes placed in an approximately tetrahedral configuration around the heart, the specialized lead adaptor, and the conventional pulse generator allow for pacing from the pulse generator to electrically activate four areas of the heart.

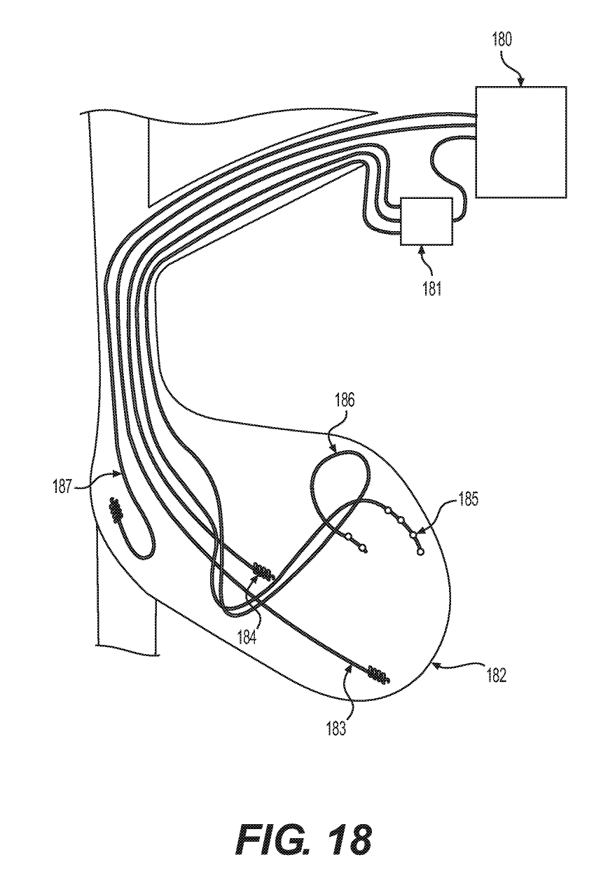

FIG. 18 is an exemplary configuration for pacing in an approximately tetrahedral configuration around the heart with a conventional ICD 180, conventional leads, and an adaptor 181 for a patient requiring resynchronization in sinus rhythm. In this example, an atrial lead 187 is positioned with its tip in the right atrial appendage and connected to the atrial port of the ICD. An active fixation ICD lead 183 is positioned with its tip deployed the RV apex and connected to the DF-4 port of an ICD. RV2 184 is an active fixation bipolar pacing lead with its tip deployed at the RV base. LV1 185 is a quadrapolar LV lead positioned in a lateral branch of the left ventricle. LV2 186 is a bipolar LV lead positioned in the anterior intraventricular vein. RV2, LV1, and LV2 are connected to an adaptor and that adaptor is connected to the IS-4 port of an ICD. The adaptor results in (1) the tip electrode of LV1 being electrically connected to pole 1 of the adaptor IS-4 connector, (2) electrode 3 of LV1 being electrically connected to pole 2 of the adaptor IS-4 connector, (3) the tip electrode of LV2 being electrically connected to pole 3 of the adaptor IS-4 connector, and (4) the tip electrode of RV2 being electrically connected to pole 4 of the adaptor IS-4 connector. The ICD paces in a DDD mode in the following manner. First the atrium is paced or sensed by the atrial lead connected to the atrial port of the ICD. After an appropriate AV delay (80-200 ms), a cathodal pacing stimulus is delivered to pole 1 or pole 2 of the IS-4 port of the ICD with the reference anode being the other pole (bipolar pacing), the ICD RV coil, or the pulse generator. Next, after the reduced or even minimal programmable delay (0-10 ms), a cathodal pacing stimulus is delivered to pole 3 and an anodal pacing stimulus is delivered to pole 4 of the IS-4 connector resulting in a cathodal stimulus to the LV2 tip electrode and an anodal stimulus to the RV2 tip electrode. The cathodal stimulus threshold of an LV2 is expected to be similar to an anodal stimulus threshold of RV2 so that a single bipolar pulse from the pulse generator should be able to electrically capture both sites with and output of 2-3 V at 0.4-1.0 ms. After the next reduced or even minimal programmable delay of the pulse generator (0-10 ms), a cathodal stimulus is delivered to the ICD lead tip. Thus, pacing in an approximately tetrahedral pattern around the left ventricle is accomplished.

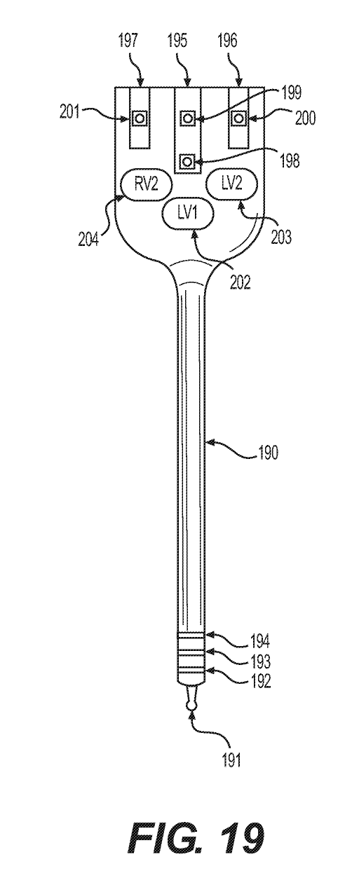

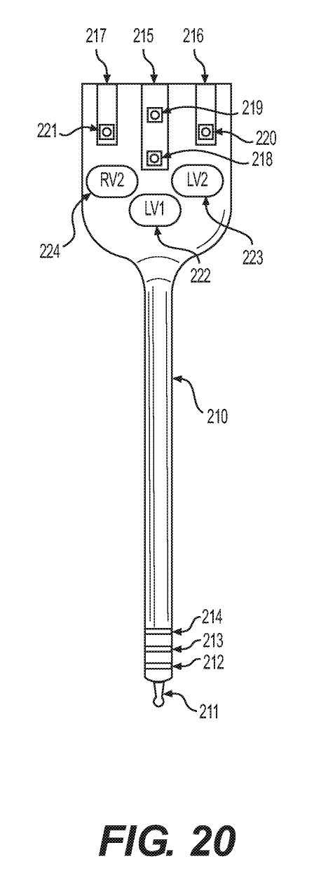

A specific description of an adaptor described in FIG. 18 is further illustrated in FIG. 19 190. This adaptor electrically connects three separate leads to an IS-4 connector. The IS-4 connector consists of tip electrode 191, ring electrode 2 192, ring electrode 3 193, and ring electrode 4 194. The adaptor has three ports for connecting three leads. An LV lead 185 positioned in a branch of the coronary sinus is referred to as the LV1 lead and is a quadrapolar lead with an IS-4 connector. The LV1 lead connects to the adaptor port 195 which is associated with the label ‘LV1’ 202 using two hex screw connectors 198 and 199. The first hex screw connector 198 is used to connect the first electrode (‘tip’) of the LV1 lead IS-4 connector to a wire that is electrically isolated and connected to the first electrode (‘tip’) of the adaptor IS-4 connector 191. The second hex screw connector 199 is used to connect the third electrode of the LV1 lead IS-4 connector to a wire that is electrically isolated and connected to the second electrode of the adaptor IS-4 connector 192. An LV lead positioned in the anterior interventricular vein 186 (FIG. 18) is referred to as the LV2 lead and is a bipolar lead with an IS-1 connector. The LV2 lead connects to the adaptor port 196 which is associated with the label ‘LV2’ 203 using a hex screw connector 200. The third hex screw connector 200 is used to connect the first electrode (‘tip’) of the LV2 lead IS-1 connector to a wire that is electrically isolated and connected to the third electrode of the adaptor IS-4 connector 193. A right ventricular lead positioned in basal portion of the right ventricle (FIG. 18 184 is referred to as the RV2 lead and is a bipolar lead with an IS-1 connector. The RV2 lead connects to the adaptor port 197 which is associated with the label ‘RV2’ 204 using a hex screw connector 201. The fourth hex screw connector 201 is used to connect the first electrode (‘tip’) of the RV2 lead IS-1 connector to a wire that is electrically isolated and connected to the fourth electrode of the adaptor IS-4 connector 194.

An alternative configuration to using a quadrapolar LV lead with an IS-4 connector in the branch of the coronary sinus is to use a bipolar LV lead with an IS-1 connector. This may be necessary for some types of cardiac anatomy. Since this is a different connector, a different adaptor would be necessary. An alternative adaptor to that described in FIG. 18 is further illustrated in FIG. This adaptor electrically connects three separate leads to an IS-4 connector. The IS-4 connector consists of tip electrode (element 211, ring electrode 2 212, ring electrode 3 213, and ring electrode 4 214. The adaptor has three ports for connecting three leads. An LV lead positioned in a branch of the coronary sinus (FIG. 18, 185) is referred to as the LV1 lead and is a bipolar lead with an IS-1 connector. The LV1 lead connects to the adaptor port (element 215) which is associated with the label ‘LV1’ 222 using two hex screw connectors elements 218 and 219. The first hex screw connector 218 is used to connect the first electrode (‘tip’) of the LV1 lead IS-1 connector to a wire that is electrically isolated and connected to the first electrode (‘tip’) of the adaptor IS-4 connector 211. The second hex screw connector 219 is used to connect the ring electrode of the LV1 lead IS-1 connector to a wire that is electrically isolated and connected to the second electrode of the adaptor IS-4 connector 212. An LV lead positioned in the anterior interventricular vein (FIG. 18, 186 is referred to as the LV2 lead and is a bipolar lead with an IS-1 connector. The LV2 lead connects to the adaptor port 216 which is associated with the label ‘LV2’ 223 using a hex screw connector 220. The third hex screw connector 220 is used to connect the first electrode (‘tip’) of the LV2 lead IS-1 connector to a wire that is electrically isolated and connected to the third electrode of the adaptor IS-4 connector 213. A right ventricular lead positioned in basal portion of the right ventricle (FIG. 18, 184 is referred to as the RV2 lead and is a bipolar lead with an IS-1 connector. The RV2 lead connects to the adaptor port 217 which is associated with the label ‘RV2’ 224 using a hex screw connector 221. The fourth hex screw connector 221 is used to connect the first electrode (‘tip’) of the RV2 lead IS-1 connector to a wire that is electrically isolated and connected to the fourth electrode of the adaptor IS-4 connector 214.

The limitation of this method is the complexity of lead deployment, number of leads, and need for an adaptor. The battery drain with pacing will be similar to current battery drain with convention ICDs. The inclusion of the leads and pacing method described in this invention will further improve pacing in a tetrahedral configuration by improving lead delivery and reducing current drain on the pulse generator battery.

One skilled in the art will recognize that multiple configurations pulse generators, pacing leads, and adaptors are possible to result in pacing in an approximately tetrahedral pattern around the heart. For example, if a patient had chronic atrial fibrillation the atrial port may be utilized for pacing the ventricle. RV2 and LV1 may be connected to a serial Y-adaptor with the LV1 tip electrically connected to the adaptor IS-1 tip and the RV2 tip electrically connected to the adaptor IS-2 ring. Pacing from the atrial port now results in cathodal stimulation at LV1 and anodal stimulation at RV2 with a single pulse. The LV1 lead is connected to the IS-4 port and the ICD lead is connected to the DF-4 port of the ICD. The pulse generator is again programmed to the reduced or even minimal programmable delays between the pacing pulses to result in pacing in a tetrahedral configuration around the heart.

If a pacemaker rather than ICD is desired as the pulse generator, then the ICD lead may be substituted for an active or passive fixation pacing lead and connected to the RV port of the pacemaker. If the patient is in sinus or an atrial paced rhythm, the atrial, RV2, LV1, and LV2 leads are implanted and adapted in the same manner as described for FIG. 18. If the patient is in atrial fibrillation, then a bipolar pacing lead may be utilized rather than an ICD lead and the atrial IS-1 port and LV IS-4 port are then utilized in the same manner as described for tetrahedral pacing with and ICD.

Thus, there are four combinations for pacing in a tetrahedral pattern around the left ventricle: (1) sinus or atrial paced rhythm with pacing in a tetrahedral configuration using conventional leads, an adaptor, and an ICD; (2) atrial fibrillation with pacing in a tetrahedral configuration using conventional leads, an adaptor, and an ICD; (3) sinus or atrial paced rhythm with pacing in a tetrahedral configuration using conventional leads, an adaptor, and a pacemaker; and (4) atrial fibrillation with pacing in a tetrahedral configuration using conventional leads, an adaptor, and a pacemaker. Additionally, if the LV1 lead is chosen to be a bipolar lead rather than a quadrapolar lead, then a different adaptor may be chosen for that configuration.

An additional embodiment of the present disclosures, therefore, provides a pacing system comprising an adaptor comprising electrically connecting electrodes disposed on three separate pacing leads to a single electrical connection with four electrodes. In some aspects, the single electrical connection with four electrodes meets IS-4 standards. In other aspects, he three pacing leads are two IS-1 leads and one IS-4 lead. In still other aspects, the three pacing leads are IS-1 leads.

1. A multi-site right ventricular lead, comprising:
a distal section that is elongate along a central axis,
the distal section having a terminus, and
the distal section having a distal fixation that extends away from the distal terminus; and
a proximal section that is elongate along the central axis and in electrical communication with the distal section,
the proximal section having at least one electrode and having an end adapted to be connected to a pulse generator.

2. The multi-site right ventricular lead of claim 1, wherein the distal section further comprises a distal electrode.

3. The multi-site right ventricular lead of claim 1, wherein the distal fixation comprises a helical electrode.

4. The multi-site right ventricular lead of claim 1, wherein the distal section further comprises a distal coil.

5. A left ventricular lead system, comprising:
an outer lead that is elongate along a central axis,
the outer lead having a first end adapted to be connected to a pulse generator, and
the outer lead having a second end having at least one electrode;
a first lumen that is disposed along or parallel to the central axis of the outer lead and between the first end and the second end of the outer lead and being configured to receive a connector,
the first lumen further having a receiving port; and
a second lumen configured to house a portion of the inner lead, the second lumen being disposed along or parallel to the central axis;
an inner lead having a first end adapted to be connected to the receiving port and having a second end having at least one electrode; and
a securing mechanism disposed at or near the second lumen,
the securing mechanism being configured to prevent the inner lead from moving in the second lumen once activated.

6. A method of heart stimulation employing a multi-electrode array in contact with the heart, comprising:
delivering a first pulse to a first set of electrodes in electrical communication with heart tissue,
the first set of electrodes comprising at least one cathodal electrode and at least one anodal electrode;
delivering a second pulse to a second set of electrodes,
the second set of electrodes comprising at least one cathodal electrode and at least one anodal electrode,
wherein the first set of electrodes and the second set of electrodes comprise overlapping electrodes having at least one electrode in common, the first and second pulses received by the at least one electrode in common being of opposite phases, and
wherein at least the first pulse or the second pulse effects a pacing pulse between its at least one cathodal and at least one anodal electrodes.

7. The method of claim 6, wherein the multi-electrode array comprises two electrodes.

8. The method of claim 7, further comprising delivering additional pulses to the two electrode array, wherein each electrode receives an opposite phase pulse compared to an immediately previous pulse.

9. The method of claim 6, further comprising delivering additional pulses to one or more additional sets of electrodes, each one or more additional sets of electrodes comprising at least one cathodal electrode and at least one anodal electrode, wherein each one or more additional set of electrodes comprises at least one overlapping electrode in common from a set of electrodes receiving an immediately previous pulse.

10. The method of claim 9, wherein pulses are delivered in a sequence such that each pulse is delivered to a unique electrode.

11. The method of claim 10, wherein the overlapping electrodes of the array have an ascending order of thresholds and the pulses are delivered in a sequence according to the ascending order of thresholds.

12. The method of claim 9, wherein the pulses are delivered in a sequence that reduces energy expenditure from a power source.

13. The method of claim 8, wherein multi-electrode array comprises four electrodes.

14. The method of claim 13, wherein the electrodes are arranged around, and in contact with, the heart in a tetrahedral fashion.

15. The method of claim 8, wherein the multi-electrode array comprises more than four electrodes.

16. The method of claim 15, wherein the more than four electrodes are arranged approximately equidistantly around, and in contact with, the heart.

17. A method of heart stimulation, employing a multi-electrode array in contact with the heart, the multi-electrode array having at least three electrodes, comprising:
a) delivering a first pulse to the first electrode and a first pulse to the second electrode so as to effect a pacing impulse between the first and second electrodes,
the first pulse to the first electrode having a polarity opposite to the first pulse to the second electrode; and
b) delivering a second pulse to the second electrode and a second pulse to the third electrode so as to effect a pacing impulse between the second and third electrodes,
the second pulse to the second electrode having a polarity opposite to the first pulse to the second electrode;

18. The method of claim 17, wherein the array comprises more than three electrodes and wherein steps a) and b) are performed sequentially with the more than three electrodes.

19. The method of claim 17, wherein steps a) and b) are controllable performed iteratively over all electrodes present in the array.

20. The method of claim 17, wherein the first pulse delivered to the first electrode is a depolarizing cathodal pulse.

21. The method of claim 17, wherein multi-electrode array comprises four electrodes.

22. The method of claim 21, wherein the electrodes are arranged around, and in contact with, the heart in a tetrahedral fashion.

23. The method of claim 17, wherein the multi-electrode array comprises more than four electrodes.

24. The method of claim 23, wherein the more than four electrodes are arranged approximately equidistantly around, and in contact with, the heart.

25. A method for sensing cardiac electrical activation, comprising:
positioning a distal electrode of a right ventricle lead in the apex region of a right ventricle of a subject or to a right ventricular septum;
positioning a proximal electrode of the right ventricular lead in the a basilar portion of the right ventricle;
employing the electrodes of the right ventricle lead to collect electrical signals generated by the right ventricle;
positioning a first electrode of a left ventricular lead in a first branch of the coronary sinus of a left ventricle;
positioning a second electrode of the left ventricle lead in a second branch of the coronary sinus of the left ventricle; and
employing the electrodes of the left ventricular lead to collect one or more electrical signals generated by the left ventricle.

26. The method of claim 25, further comprising converting the collected electrical signals into electrograms.

27. The method of claim 26, further comprising constructing a model of the subject's ventricular tachycardia based at least in part on the electrograms and on the subject's specific anatomy.

28. The method of claim 25, further comprising storing the electrical signals in a database.

29. The method of claim 25, further comprising applying overlapping multiphasic stimulation when the collected signals indicate a premature ventricular beat.

30. The method of claim 25 wherein the electrodes are arranged around, and in contact with, the heart in a tetrahedral fashion.

31. The method of claim 29, wherein the electrodes are arranged approximately equidistantly around, and in contact with, the heart.

31. (canceled)

32. The method of claim 25, further comprising positioning at least one additional electrode.

33. The method of claim 32, wherein the electrodes are arranged approximately equidistantly around, and in contact with, the heart.

34. A method of cardiac resynchronization therapy, comprising:
positioning a distal electrode of a right ventricle lead in the region of the apex of a right ventricle of a subject or distal to a right ventricular septum;
positioning a proximal electrode of the right ventricular lead in the region of the a basilar portion of the right ventricle;
positioning a first electrode of a left ventricular lead in a first branch of the coronary sinus of a left ventricle;
positioning a second electrode of the left ventricle lead in a second branch of the coronary sinus of the left ventricle; wherein the electrodes are electrically connected with a pulse generator by a lead comprising a conductor and that conductor is electrically isolated by insulation;
delivering a first pulse to a first set of electrodes, the first set comprising at least one cathodal electrode and at least one anodal electrode; delivering a second pulse to a second set of electrodes, the second set of electrodes comprising at least one cathodal electrode and at least one anodal electrode,
wherein the first set of electrodes and the second set of electrodes comprise overlapping electrodes having at least one electrode in common, the first and second pulses received by the at least one electrode in common being opposite phases, and
wherein at least the first pulse or the second pulse effects a pacing impulse between its at least one cathodal and at least one anodal electrodes.

35. The method of claim 34, further comprising positioning additional sets of electrodes and delivering pulses to additional sets of electrodes, each additional set comprising at least one cathodal electrode and at least one anodal electrode, wherein each additional set comprises overlapping electrodes having at least one electrode in common from a set of electrodes receiving an immediately previous pulse.

36. The method of claim 35, wherein pulses are delivered in a sequence such that each pulse is delivered to a unique electrode.

37. The method of claim 34, wherein the electrodes of the array have an ascending order of thresholds and the pulses are delivered in a sequence according to the ascending order of thresholds.

38. The method of claim 34, wherein the pulses are delivered in a sequence that reduces energy expenditure from a power source.

39. The method of claim 34, further comprising a leadless pacemaker having an accelerometer to sense motion, rate smoothing, and to appropriately time pacing from the leadless pacemaker to the tetra-pacing system.

40. The method of claim 34, further comprising delivering the pulses about simultaneously.

41. The method of claim 34, wherein the sequence of pulses is delivered such that each pair of electrodes receiving a pulse includes a first electrode that received an immediately previous pulse and a second electrode that did not receive the immediately previous pulse.

42. The method of claim 41, wherein the first electrode receiving a cathodal phase of a pulse received an anodal pulse during an immediately previous pulse.

43. The method of claim 34, wherein the electrodes are arranged around, and in contact with, the heart in a tetrahedral fashion.

44. The method of claim 43, wherein the electrodes are arranged approximately equidistantly around, and in contact with, the heart.

45. A method of applying a pulse sequence to a multi-electrode array, comprising:
determining a unipolar capture threshold for each electrode in the array;
determining an electrode pacing order, wherein the electrode pacing order is in ascending order of the unipolar capture thresholds of the electrodes;
determining a threshold for a pacing orientation for each pulse delivered to the array, wherein each pulse is delivered to a first electrode and a next electrode in the electrode pacing order;
designing a pulse sequence for the array; and
applying the pulse sequence to the array.

46. A method of applying an overlapping multiphasic stimulation pulsing sequence in a multi-electrode array, comprising:
selecting a pulse unit;
assessing a unipolar capture threshold for each electrode in the array for a given pulse width, orientation, and number of pulses;
designing a pulse sequence that requires a reduced number of pulses; and
applying the pulse sequence to a cardiac resynchronization system.

47. A pacing system comprising:
an adaptor comprising electrically connecting electrodes disposed on three separate pacing leads to a single electrical connection with four electrodes.

48. The pacing system of claim 47, wherein the single electrical connection with four electrodes meets IS-4 standards.

49. The pacing system of claim 47, wherein the three pacing leads are two IS-1 leads and one IS-4 lead.

50. The pacing system of claim 47, wherein the three pacing leads are IS-1 leads.
